(19) United States
(12) Patent Application Publication
(10) Pub. No.: US 2021/0085736 A1
(43) Pub. Date: Mar. 25, 2021

(54) IMMUNO-ONCOLYTIC MODIFIED VACCINIA TIAN TAN VIRUS AND METHODS OF TREATING CANCER

(71) Applicant: The University of Hong Kong, Hong Kong (CN)

(72) Inventors: Zhiwei Chen, Hong Kong (CN); Zhiwu Tan, Hong Kong (CN)

(21) Appl. No.: 17/048,297

(22) PCT Filed: Apr. 19, 2019
(86) PCT No.: PCT/IB2019/000525

§ 371 Date: Oct. 16, 2020

Related U.S. Application Data
(60) Provisional application No. 62/660,546, filed on Apr. 20, 2018.
(60) Provisional application No. 62/687,531, filed on Jun. 20, 2018.

(51) Int. Cl. A61K 35/768; A61P 35/00

(52) U.S. Cl. CPC A61K 35/768; A61K 45/06; A61P 35/00

(57) ABSTRACT

The invention pertains to methods of treating a cancer in a subject by administering to the subject a combination of an oncolytic virus and a therapy that induces depletion of tumor-induced bone marrow myeloid-derived suppressor cells of polymorphonuclear type (PMN-MDSCs). In certain preferred embodiments, the oncolytic virus is a replication incompetent modified vaccinia TianTan (MVTT) virus having a deletion of the viral M1L-K2L genes. In other preferred embodiments, the therapy that induces depletion of tumor-induced PMN-MDSCs comprises administering an antibody against Ly6G, for example 1A8. The cancer therapies of the invention can be administered in combination with one or more additional anti-cancer therapies. Preferred additional anti-cancer therapy is an immunotherapy, such as administering a check-point inhibitor.

### CROSS-REFERENCE TO RELATED APPLICATIONS

This application claims the benefit of U.S. Provisional Patent Application Ser. Nos. 62/660,546, filed Apr. 20, 2018, and 62/687,531, filed Jun. 20, 2018, which are hereby incorporated by reference in their entirety including any tables, figures, or drawings.

### BACKGROUND OF THE INVENTION

Mesothelioma is an asbestos-associated malignant form of cancer, which often has a poor prognosis in humans. The current standard of care for this life-threatening malignancy only achieves suboptimal improvements in patient survival. Harnessing the host immune system to eradicate malignant cells has become a clinical strategy in cancer immunotherapy. Although immune checkpoint inhibitors have improved the therapeutic efficacy in certain cancers, their effects are unsatisfactory in patients with mesothelioma. Therefore, novel strategies are needed for treating mesothelioma. Recently, oncolytic virotherapy has emerged as a promising cancer immunotherapy for the treatment of solid tumors including malignant mesothelioma. However, the mechanisms underlying the limited virotherapeutic efficacy remains elusive.

Direct virus-mediated oncolysis of cancer cells is one of the major mechanisms of oncolytic virotherapy. During oncolysis, danger-associated molecular patterns (DAMPs) and pathogen associated molecular patterns (PAMPs) are released into the tumor microenvironment (TME), which can modulate the immunogenicity of released tumor antigens by creating an immune-activating environment and subsequently eliciting or reinforcing tumor-reactive T cell responses. The crucial role of adaptive T cell immunity in oncolytic virotherapy has been demonstrated in both preclinical and clinical studies. However, TME is often an immunosuppressive environment that inhibits the activation of tumor-reactive T cells by inducing tolerogenic dendritic cells (DCs) and CD25⁺Foxp3⁺ regulatory T lymphocytes (Tregs). Bone marrow myeloid-derived suppressor cells (MDSCs) in the TME can dampen the responsiveness of cytotoxic T lymphocytes (CTLs), leading to limited efficacy in patients, especially when the TME is highly immunosuppressive. Because T cell immunity is indispensable for the efficacy of oncolytic virotherapy, the better understanding of restrictive mechanisms in the TME is particularly important for improving the clinical outcomes of oncolytic virotherapies.

MDSCs represent one of the major immunosuppressive populations in the TME and a major obstacle to the effectiveness of cancer immunotherapy. In malignant mesothelioma models, MDSCs expand quickly with the development of tumor lesions and contribute to the inhibition of tumor-reactive CTL responses. Consistently, decreased numbers of MDSCs in the TME are likely associated with the generation of antigen-specific CTL responses and therapeutic efficacy during oncolytic virotherapy in patients. MDSCs can be monocytic (M) or polymophonuclear (PMN). Targeting the COX-2-PGE2 pathway during vaccinia virotherapy is capable of decreasing PMN-MDSC levels while increasing antitumor CTL responses. Moreover, an earlier study using the COX-2 inhibitor celecoxib improved DC-based immunotherapy against mesothelioma by reducing the PMN-MDSC frequency. While these studies indicate the critical role of PMN-MDSCs in cancer immunotherapy, curing established tumors has rarely been observed. To date, the mechanism underlying MDSCs accumulation in the TME, the functional difference between MDSC subsets, and their impact on eliciting antitumor CTLs during oncolytic virotherapy remain incompletely understood.

### BRIEF SUMMARY OF THE INVENTION

In certain embodiments, the invention provides methods of treating a cancer in a subject by administering to the subject a combination of an oncolytic virus and a therapy that induces depletion of tumor-induced PMN-MDSCs. In preferred embodiments, the oncolytic virus is a replication incompetent modified vaccinia TianTan (MVTT) virus having a deletion of the viral M1L-K2L genes. In other preferred embodiments, the therapy that induces depletion of tumor-induced PMN-MDSCs comprises administering an antibody against Ly6G, for example 1A8. The cancer therapies of the invention can be administered in combination with one or more additional anti-cancer therapies. Preferred additional anti-cancer therapy is an immunotherapy, such as administering a check-point inhibitor.

### BRIEF DESCRIPTION OF THE DRAWINGS

FIGS. 1A-1E show generation of recombinant MVTT virus that encodes two detection markers, HIV-1 p24 and RFP. (A) Schematic representation of vaccinia shuttle vector pZCxz encoding both HIV-1 p24 and HcRed. Expression of each protein is driven by a different promoter. (B) AB1 cells were infected with the recombinant MVTT for 24 hours. HcRed signals were acquired with fluorescent microscopy. BF, Bright Field. (C) Western blot analysis of viral protein expression in AB1 cells after recombinant MVTT infection. Anti-p24 antibody (clone: 183-H12-5C) was used to detect the presence of foreign protein as indicated by the arrow. GAPDH is an internal control to indicate that equal amount of proteins was loaded in each lane. (D) AB1 cells were seeded in 24-well plate at a density of 2×10⁵ cells/well. 24 hours later, cells were infected with 0.2 multiplicity of infection (MOI) recombinant MVTT virus. Cells were harvested at three indicated time points and percentage of HcRed⁺ AB1 cells were analyzed using flow cytometry. (E) Culture supernatant after recombinant MVTT viral infection was collected from AB1 cells at different time points and viral particles released into the supernatant were measured.

FIGS. 2A-2E show MVTT-mediated oncolysis of AB1 cells leading to exposure of CRT as well as release of ATP and HMGB1. (A) AB1 cell viability upon infection with 0.2 MOI recombinant MVTT. CRT expression on the AB1 cells were detected by anti-CRT antibody and analyzed either by flow cytometric analysis (B) or western blotting (C). (3-actin is an internal control showing that the same amount of proteins was used for the analysis. (D) Western blot analysis of released HMGB1 in the culture supernatant after MVTT virus infection. (E) Released ATP level in the culture supernatant.

FIGS. 3A-3F show that oncolysis of AB1 mesothelioma by recombinant MVTT virus did not induce immunogenic death of tumor cells. (A) Schematic representation of therapeutic study on AB1 tumor-bearing mice using different doses of MVTT. Solid AB1 mesothelioma was established with subcutaneous inoculation of 5×10⁵ AB1 cells 7 days before treatment. In high-dose group, 1×10⁸ PFU MVTT virus per dose was delivered intra-tumorally (i.t.) every 2 days for 5 times, while in medium-dose group 1×10⁷ PFU each injection was given i.t. for 4 times and 2 times for low-dose group. (B) Tumor volume was measured overtime with a caliper. (C) Individual tumor growth curve in each group. Every line represents one mouse. (D) Survival curve, taken as time to tumor length >15 mm, was determined by caliper measurement. (E) T cell responses in splenocytes of tumor-free or AB1 tumor-bearing mice. Secreted IFN-γ was quantified by ELlspot assay after ex vivo stimulation of splenocytes with gp70-AH1, TWIST1 or an irrelevant antigen, OVA. Only one tumor-free mouse had strong responses against gp70-AH1 epitope, as indicated by the arrow. (F) CTL assay for CD3⁺ T cells isolated from tumor-free mice. The grey line represents CTL activity of CD3⁺ T cells from the mouse with strong AH1 responses. P=0.08, compared to PBS group.

FIGS. 4A-4F show accumulation of PMN-MDSCs in tumors after intra-tumoral MVTT treatment. (A) Percentage of total MDSCs in the spleen and tumor (left panel) and absolute cell number of MDSCs in the tumor (right panel). Numbers of MDSCs per milligram of tumor at indicated time points were calculated. (B) Representative dot plots showing population of PMN-MDSCs and M-MDSCs within CD11b⁺ cells in the spleen and tumor. Numbers indicating cell proportions. (C) Percentages of MDSC subsets were calculated with M-MDSCs (left panel) and PMN-MDSCs (right panel). (D) Absolute cell number of M-MDSCs and PMN-MDSCs in the tumor. Numbers of MDSC subsets per milligram of tumor at indicated time points were calculated. (E) Percentage of CD4⁺ Treg in the spleen and tumor (left panel) and absolute cell number of CD4⁺ Treg in the tumor was also shown (right panel). (F) Percentage of NK cells in the spleen and tumor (left panel) and absolute cell number of NK cells in the tumor (right panel).

FIGS. 5A-5F show trafficking of PMN-MDSCs to the tumor site after intra-tumoral MVTT treatment. (A) Flow cytometric analysis of chemokine receptors expression on different MDSCs subsets from AB1 tumor-bearing mice. Representative histogram plots are shown; shaded region represents isotype control. Expression of C—X—C chemokines (B) and C—C chemokines (C) in the tumor after MVTT treatment. (D) Frequencies (left panel) and absolute number (right panel) of CFSE labelled MDSCs in both spleen and tumor 24 hours after MVTT treatment. (E) M-MDSCs and PMN-MDSCs cell subsets in the tumor 24 hours after MVTT treatment. Representative dot plots are shown with numbers indicating gated cell proportions to total singlets. (F) Changes in the ratio of PMN-MDSCs proportion over M-MDSCs proportion were analyzed (left panel). PMN-/M-MDSCs ratio measured before adoptive transfer was shown as baseline. Changes in the absolute numbers of M-MDSCs and PMN-MDSCs in the tumor are shown (right panel).

FIG. 6A-6D show disrupting PMN-MDSCs tumor trafficking after MVTT treatment. (A) Representative dot plots gated on CD11b⁺ cells showing population of PMN-MDSCs and M-MDSCs in the spleen and tumor 2 days and 4 days after receiving i.t. injection of 100 μg of either 1A8 or anti-rat IgG₂ₐ (clone: 2A3) isotype control. Numbers within dot plots represent cell proportions in the gate. (B) Percentages of MDSCs subsets were calculated with PMN-MDSCs (left panel) and M-MDSCs (right panel). (C) Representative dot plots showing population of PMN-MDSCs and M-MDSCs in the spleen and tumor 2 days and 4 days after combination treatment. 100 μg of either 1A8 or isotype 2A3 were combined with 1×10⁷ PFU MVTT and i.t. injected into AB1 mesothelioma. (D) Analysis of changes in MDSC subsets with PMN-MDSCs (left panel) and M-MDSCs (right panel).

FIGS. 7A-7K show combination of oncolysis and PMN-MDSC depletion restored antitumor T cell immunity for tumor elimination. (A) Schematic representation of treatment schedule. 5×10⁵ AB1 cells were subcutaneously (s.c.) inoculated into Balb/c mice and left to grow for 7 days, following i.t. administration of MVTT, 1A8 antibody, MVTT+1A8 combination or PBS control. An additional treatment was scheduled at day 9 in each group. Tumor growth (B) and survival curve (C) in mice were calculated. 40 days after tumor ablation, protected mice in combination treatment group were re-challenged and measured for tumor growth (D) with representative bioluminescence images of AB1-Luc tumors (E). (F) T cell responses in splenocytes measured by ELlspot assay. (G) In vitro cytotoxic activity of CD3⁺ T cells in each group, or CD4⁺ and CD8⁺ T cells from MVTT+1A8 treated group, towards AB1 cells at different effector:target (E:T) ratios. (H) Schematic representation for T cell depletion with 2 times of MVTT+1A8 combination therapy. AB1-Luc tumor growth (I) and survival curve (J) of MVTT+1A8 treated mice without CD4⁺ T cells (YTS191.1), CD8⁺ T cells (YTS169.4) or AB1-Luc tumor-bearing mice receiving isotype control (LTF-2) only. (K) Representative bioluminescence images of AB1-Luc tumors in T cell depletion groups.

FIGS. 8A-8F show that PMN-MDSCs prevent the induction of antitumor T cell immunity by restricting DC activation. (A) Cytokine production following incubation of CD3⁺ T cells with antigen-pulsed BMDCs. BMDCs were pulsed with rMVTT-treated AB1 cell supernatants overnight, following washing with culture medium. Then, purified CD3⁺ T cells were added and culture supernatants were collected for analysis of cytokine production. Anti-CRT antibody or isotype control was present in several of the cultures during antigen-pulsing. Naïve, purified CD3⁺ T cells from naïve BALB/c mice. (B) Proliferation of CFSE-labelled CD3⁺ T cells after co-culture with antigen-pulsed BMDCs. Representative histograms are shown with numbers in each plot indicating proliferating populations. (C) Expression of CD80 and CD86 on BMDCs pulsed with culture medium (Unstimulated) or LPS. Purified PMN-MDSCs or M-MDSCs were labelled with CFSE and were present in the culture at a ratio of 2:1 with BMDCs. Graphs from (A) to (C) show cumulative data from two separate experiments. (D) Frequencies of IL-10⁺ and TGF-β1⁺ PMN-MDSCs and M-MDSCs. Representative dot plots from 3 independent experiments are shown with numbers indicating positive cell populations in each gate. (E) Production of IL-10 was enhanced by crosstalk between PMN-MDSCs and BMDCs. 5×10⁴ purified PMN-MDSCs or M-MDSCs were present in the culture with or without 1×10⁵ BMDCs (BMDC:MDSC=1:2). Supernatant were collected at 4 days post incubation and measured for cytokine production. (F) Expression of CD80 and CD86 on LPS-activated BMDCs in the presence of IL-10 receptor blocking antibody or isotype control. Purified PMN-MDSCs or M-MDSCs were labelled with CFSE and were present in the culture at a ratio of 2:1 with BMDCs. IL-10 receptor was blocked by anti-IL-10R antibody (5 μg/ml) before BMDCs were stimulated with 100 ng/ml LPS. Graphs from (E) to (F) show representative data from two separate experiments.

FIGS. 9A-9C show that combination therapy significantly inhibited B16F10 melanoma growth in C57BL/6 mice. C57BL/6 mice were implanted s.c. with 5×10⁵ B16F10-Luc cells 7 days before treatment. Tumor growth (A), survival curve (B) and T cell responses of splenocytes (C) at their endpoint were shown.

FIGS. 10A-10E show that MVTT treatment recruited PMN-MDSCs into the TME. (A) Expression of HcRed in established AB1 mesothelioma tumors after rMVTT treatment. Overlay of representative light and fluorescent images of HcRed in the tumor with or without rMVTT injection (left panel). Fluorescence images were acquired using an IVIS Spectrum instrument. The color bar indicates the fluorescence radiant efficiency multiplied by 10⁷. Representative images are shown. HcRed fluorescent signals from tumors were calculated (right panel). (B) Immunohistochemistry of vaccinia virus proteins in AB1 tumors 2 days post rMVTT injection. AB1 tumor sections were stained with hematoxylin & eosin (H&E) (left panel) or stained for vaccinia virus proteins (Green) using a commercially obtained rabbit anti-vaccinia virus antibody (WR, Access Biomedical) and Hoechst 33258 staining (blue) (right panel). Representative images are shown. Dotted line shows the boundary between infected and un-infected tumor tissue. (C) Gating strategies for flow cytometric scatter plots showing identification of MDSC subsets, NK cells, and CD4⁺ Tregs, as well as PD1⁺/Tim3⁺ CD3⁺ T cells. (D) Frequencies (left panel) and absolute numbers (right panel) of CD3⁺ T cells in the tumor. (E) Frequencies of PD1⁺ CD3⁺ T cells (left panel) and Tim3⁺ CD3⁺ T cells (right panel) in the spleen and tumor.

FIG. 11 shows flow cytometric analysis of CFSE-labelled MDSCs. Adoptively transferred MDSCs accumulated at the tumor site 24 hours after rMVTT treatment in representative mice. Numbers within dot plots represent CFSE⁺ cell proportions relative to total singlets.

FIGS. 12A-12H show preferential depletion of MDSC subsets by antibody and peptibody treatment. (A) Schematic representation of H6/G3-pep-encoding plasmid. IL2ss, IL2 secretary signal. The binding affinity of H6-pep, G3-pep, or peptibody without the 12-merspecific sequence (control-pep) was measured by flow cytometry. Splenocytes from AB1-tumor bearing mice were incubated with 2 μg of peptibody following detection with anti-mouse IgG2b AF568. (B) Representative dot plots gated on CD11b⁺ cells are shown with numbers indicating cell proportions. (C) Representative histogram plots gated on CD11b⁺ cells are shown with pep-H6 (dashed line), G3-pep (solid line), or control-pep (shaded histogram) staining. (D) Percentages of total MDSCs in the spleen and tumor after i.t administration of 100 μg of 1A8, H6-pep, or 2A3 isotype control. Changes in PMN-MDSC and M-MDSC frequencies after i.t. H6-pep treatment were shown with representative dot plots (E) and were analyzed (F). After i.t co-administration of 1×10⁷ PFU rMVTT and 100 μg of H6-pep, changes in the PMN-MDSC and M-MDSC frequencies are shown (G) and were analyzed (H).

FIGS. 13A-13I show depletion of PMN-MDSCs enhances MVTT treatment efficacy by inducing antitumor T cell immunity. (A) Schematic representation of the treatment schedule where one administration of either PBS, 1A8 only, combined rMVTT and 1A8, or combined rMVTT and H6-pep was given 7 days after AB1 cell inoculation. Tumor growth (B) and survival curve (C) of mice receiving one round of treatment. Tumor growth (D), survival curve (E) and T cell responses of splenocytes (F) in mice receiving 2 injections of PBS, H6-pep or combined rMVTT and H6-pep. C57BL/6 mice were implanted s.c. with 5×10⁵ B16F10-Luc cells 7 days before treatment. rMVTT, 1A8 antibody, combined rMVTT and 1A8 or PBS control were i.t. administered at day 7 and day 9. Tumor growth (G), survival curve (H) and T cell responses of splenocytes (I) at their endpoints were shown.

FIGS. 14A-14E show that PMN-MDSCs prevent the induction of antitumor T cell immunity by restricting DC activation. (A) Secretion of IL-6, IL-17A, and IL-22 in co-cultures of CD3⁺ T cells and antigen-pulsed BMDCs. Naïve, purified CD3⁺ T cells from naïve BALB/c mice. (B) Secreted cytokines in the co-culture supernatant collected 48 hours post incubation. (C) Secretion of IL-6 and TNF-α in antigen-pulsed BMDC cultures in the presence of either PMN-MDSCs or M-MDSCs at MDSC:BMDC ratios of 1:1 and 3:1. BMDCs were pulsed with rMVTT-treated AB1 cell supernatants. Data shown are representative of two independent experiments. (D) IL-10 production in tumor homogenates after rMVTT treatment. (E) Production of TNF-α and IL-12p70 in the culture supernatant in the presence of IL-10 receptor blocking antibody or isotype control. Culture supernatants were collected 48 hours post incubation and measured for cytokine secretion.

### DETAILED DISCLOSURE OF THE INVENTION

Cancer virotherapy using oncolytic viruses is a promising therapeutic strategy with demonstrated clinical benefits. Following the approval of T-vec (also known as Imlygic), a recombinant herpes simplex virus expressing the immune-activating cytokine GM-CSF for treating skin and lymph node melanoma in the USA and Europe, a variety of oncolytic viruses have progressed to clinical development. Among these, the use of ONCOS-102 adenovirus for treating malignant mesothelioma was able to induce tumor-infiltration by CD8⁺ T cells, systemic antitumor CD8⁺ T cells and Th1-type polarization in a clinical setting. Although the therapeutic effects of T-vec and ONCOS-102 are promising, only a small fraction of treated patients experienced clinical responses in these studies. Therefore, investigating how to induce potent antitumor immune responses is essential for enhancing the therapeutic efficacy of virotherapy in patients. Most of the viruses that are currently being tested in clinical trials were designed to acquire the capability to trigger immune responses. To this end, understanding the mechanism underlying the blockade and regulation of systemic antitumor immunity is critical for further improvement of oncolytic virotherapy.

Replication of the oncolytic virus in the tumor releases the danger signals CRT, HMGB1 and ATP, as well as tumor antigens for DCs, to trigger antitumor immune responses. Therefore, a combination therapy with an immunotherapy has become a useful strategy to improve the efficacy of oncolytic virotherapy in fighting various types of tumors, including malignant mesothelioma and melanoma. An immunotherapy includes augmenting host antitumor responses through the incorporation of immune activating molecules (e.g., GM-CSF), immune-regulatory drugs (e.g., cyclophosphamide), or immune checkpoint inhibitors.

In addition to the rapidly increased use of immune checkpoint inhibitors, a GM-CSF-incorporated herpes simplex virus (T-vec) has also received regulatory approval for treating patients with late-stage melanoma. Decreasing immune suppression of MDSCs and Tregs by sunitinib has been shown in clinical trials to augment anti-renal cell carcinoma immune responses during oncolytic reovirus treatment. In terms of malignant mesothelioma, the use of first-line chemotherapeutic agents (cisplatin or pemetrexed) during oncolytic adenovirus treatment has been shown to enhance virus-mediated cytotoxicity in mice.

MVTT virotherapy alone is insufficient for efficient tumor clearance. Replication of the oncolytic virus in the tumor releases the danger signals CRT, HMGB1, ATP, and tumor antigens for DCs to trigger antitumor immune responses. However, complete mesothelioma eradication was only achieved by intra-tumoral administration of extremely high doses of MVTT at multiple sites of the solid tumors, yet even in protected mice, antitumor T cell responses were rarely elicited.

The instant invention describes that virotherapy significantly expanded MDSCs in the mesothelioma TME. Expansion of MDSCs is a key immune evasion mechanism in various human cancers, such as renal cell carcinoma, squamous cell carcinoma, breast cancer, and non-small cell lung carcinoma. In mice with mesothelioma, tumors induced a rapid increase of MDSCs as early as 7 days after AB1 cell inoculation and the elimination of MDSCs during immunotherapy was closely related to tumor rejection. Expanded PMN-MDSCs in the mesothelioma TME during MVTT virotherapy were due to the production of C—X—C chemokines associated with the viral infection of tumor cells. C—X—C chemokines then preferentially recruit CXCR2⁺ PMN-MDSCs from peripheral lymphoid organs to tumor sites by chemotaxis. These results emphasize the role of the C—C and C—X—C axes in the trafficking of M-MDSCs and PMN-MDSCs, respectively.

Viral infection-recruited PMN-MDSCs were found to be responsible for either suppression of NK cells by reactive oxygen species (ROS) production or augmentation of local immune suppression by PD-L1 expression. The instant invention demonstrates that PMN-MDSCs exhibited potent immunosuppressive function against DC activation. Similar immunosuppressive effects on DCs were not found with M-MDSCs, suggesting a functional difference between these two MDSC subsets in the mesothelioma TME.

Depletion of PMN-MDSCs alone is also insufficient for efficient tumor clearance. Targeted depletion of PMN-MDSCs allowed modest CTL responses in pancreatic ductal adenocarcinoma and lung cancer models. AB1 mesothelioma in mice, however, has been recognized as a poorly immunogenic model. AB1 mesothelioma displayed similar growth kinetics in immunodeficient SCID mice compared to immunocompetent BALB/c mice.

Moreover, purified T cells from mesothelioma-bearing mice did not contain antigen-specific T cells with potent cytotoxic activity. To better define the function of PMN-MDSCs and M-MDSCs in modulating antitumor immunity, depletion experiments using anti-Ly6G or H6-pep monotherapy, respectively, were conducted. Depletion of either PMN-MDSCs or M-MDSCs did not induce any inhibitory effects on mesothelioma growth. Additionally, no measurable antitumor CTLs were detected. Therefore, depletion of MDSCs subsets alone did not promote the exposure of mesothelioma antigens to trigger DC activation. Thus, an oncolytic virotherapy is necessary to promote tumor antigen exposure and subsequent induction of systemic antitumor T cell responses.

Thus, the instant invention demonstrates that curing established mesothelioma requires a combination of an oncolytic virotherapy, such as MVTT virotherapy, and PMN-MDSC depletion, which can overcome immunosuppression despite increasing intra-tumoral M-MDSCs and potentiate DCs for the induction of potent antitumor CTLs. PMN-MDSCs play a critical role in modulating antitumor CTL responses. Using the PMN-MDSC-depleting antibody 1A8 and M-MDSC-depleting peptibody H6-pep, PMN-MDSCs but not M-MDSCs are shown to be essential for the TME to restrict the induction of tumor-reactive CTL responses during an oncolytic virotherapy, such as MVTT virotherapy.

Moreover, the combination of an oncolytic virotherapy, such as MVTT virotherapy, and depletion of PMN-MDSCs activated endogenous T cells to elicit antitumor CTLs with broad-reactive spectrum, cytolytic activity, and protective long-term memory responses. During this process, increased intra-tumoral M-MDSCs were unable to block T cell activation and antitumor CTLs.

Mechanistically, intra-tumoral PMN-MDSCs but not M-MDSCs suppressed DC activation by preventing CD80 and CD86 upregulation and IL-6, TNF-α and IL-12p70 secretion. Therefore, in addition to the suppressive effects of MDSCs on T cells, the invention describes the mechanisms by which mesothelioma-derived PMN-MDSCs exhibit immune suppressive activity on DCs. Cross-talk between PMN-MDSCs and DCs demolished antitumor immunity by increasing IL-10 production and decreasing DC activation.

Tumor-derived MDSCs upregulated IL-10 production and neutralization of IL-10 abrogated the suppressive effect of MDSCs in mouse models. Given the plasticity of the immune suppressive myeloid compartment under various tumors and infectious agents, acute phase response protein induced the expansion and polarization of IL-10-secreting tumor associated neutrophils to suppress antigen specific T cell responses in melanoma patients. Thus, IL-10-sereting PMN-MDSCs act as a barricade to protect tumors from immune surveillance. Chemotactically recruited IL-10-sereting PMN-MDSCs are critical DC suppressors to halt T cell activation during the MVTT virotherapy.

Inhibiting cell cycle-related kinase (CCRK) signaling diminished PMN-MDSC mediated immunosuppression and inhibited tumorigenicity of hepatocellular carcinoma. Therefore, an epigenetic modulatory approach targeting CCRK to specifically disrupt PMN-MDSC accumulation would be especially important in the development of combination therapy with MVTT for treating a variety of human cancers, such as mesothelioma.

Thus, the invention describes that intra-tumoral PMN-MDSCs are key suppressors of DC in the mesothelioma TME that restrict the induction of antitumor CTLs, compromising the efficacy of MVTT-based virotherapy.

Accordingly, certain embodiments of the invention provide a method of treating a cancer, such as mesothelioma, by administering a combination of an oncolytic virus and a therapy that induces depletion of tumor-induced PMN-MDSCs.

An oncolytic virus and a therapy that induces depletion of tumor-induced PMN-MDSCs can be administered simultaneously or consecutively. An oncolytic virus can be administered before or after administering a therapy that induces depletion of tumor-induced PMN-MDSCs. Co-administration of an oncolytic virus and a therapy that induces depletion of tumor-induced PMN-MDSCs can be carried out in the same or separate compositions. Separate administrations of these therapies can be performed with one or more additional agents.

When administered separately, an oncolytic virus can be administered within about one day to about seven days, preferably, within about two days to about six days, more preferably within about three to five days, and even more preferably, within about four days of administering a therapy that induces depletion of tumor-induced PMN-MDSCs. In other embodiments, when administered separately, an oncolytic virus can be administered within about 20 to 40 hours, preferably about 25 to 35 hours, even more preferably, about 30 hours, and most preferably, about 24 hours of administering a therapy that induces depletion of tumor-induced PMN-MDSCs.

In preferred embodiments, an oncolytic virus is administered before administering a therapy that induces depletion of tumor-induced PMN-MDSCs.

An oncolytic virus and a therapy that induces depletion of tumor-induced PMN-MDSCs can be administered multiple times over a period of days, for example, over two to fourteen days, more preferably, over four to twelve days, more preferably, over six to ten days, and even more preferably over about seven days.

In some embodiments, the oncolytic virus is an adenovirus, reovirus, herpes virus, picornavirus (including coxsackievirus, poliovirus, and Seneca Valley virus), paramyxovirus (including measles virus and Newcastle disease virus (NDV)), parvovirus, rhabdovirus (e.g., vesicular stomatitis virus (VSV), or vaccinia virus. The oncolytic virus can be replication competent or replication incompetent. Methods of producing replication incompetent viruses are known in the art and are within the purview of the instant invention.

In specific embodiments, the oncolytic virus is a modified vaccinia virus. Preferably, a modified vaccinia virus is a live-attenuated vaccinia virus, such as a vaccinia virus incapable of replication. In some embodiments, modified vaccinia virus is a genetically modified vaccinia virus having a deletion of one or more genes that are necessary for replication. For example, deletion of M1L-K2L genes renders a vaccinia virus incapable of replication.

An example of a modified vaccinia virus, particularly, modified vaccinia TianTan (MVTT) virus, that is suitable for use in the instant invention is described by Zhu et al. (2007), J Virol Methods; 144(1-2):17-26. The Zhu et al. reference is incorporated by reference in its entirety.

In certain embodiments, a modified vaccinia virus is a MVTT generated from vaccinia TianTan (VTT) by deleting the viral M1L-K2L genes. In other embodiments, a modified vaccinia virus is a MVTT generated from VTT by replacing the viral M1L-K2L genes with a heterologous gene, such as a gene encoding a marker fluorescent protein. Compared to the parental VTT, MVTT is 100-fold less virulent. Therefore, MVTT is an attenuated vaccinia Tian Tan vaccine vector with improved safety.

Thus, in specific embodiments, the oncolytic virus is a MVTT.

In further embodiments, the oncolytic virus is a recombinant MVTT (rMVTT). The rMVTT comprises a deletion of the viral M1L-K2L genes from a VTT and further comprises two or more heterologous genes that replace the deleted viral M1L-K2L genes. One of the two or more heterologous genes can be a gene encoding a protein label, such as a fluorescent protein or an enzyme. The fluorescent protein can be a green fluorescent protein or a red fluorescent protein. Red fluorescent protein can be HcRed or green fluorescent protein (GFP). Additional examples of fluorescent proteins are known to a person of ordinary skill in the art and such embodiments are within the purview of the invention. For example, fluorescent protein database (fpbase) is well known in the art and can be found at world-wide-web site: fpbase.org.

In further embodiments, one of the two or more heterologous genes is a gene encoding a capsid protein of a heterologous virus, preferably, p24 protein of human immunodeficiency virus (HIV). The term “heterologous virus” as used herein refers to a virus other than a VTT.

In specific embodiments, one of the two or more heterologous genes is a gene encoding a fluorescent protein and another one of the two or more heterologous genes is a gene encoding a capsid protein of a heterologous virus. Preferably, one of the two or more heterologous genes is a gene encoding HcRed and another one of the two or more heterologous genes is a gene encoding p24 of HIV.

In further embodiments, one of the two or more heterologous genes is under the control of a synapsin promoter (pSYN) and another one of the two or more heterologous genes is under the control of an H5 promoter (pH5). Preferably, one of the two or more heterologous genes is a gene encoding HcRed under the control of pH5 and another one of the two or more heterologous genes is a gene encoding p24 of HIV under the control of pSYN.MVTT readily induces DAMPs including calreticulin (CRT) exposure, HMGB1 and ATP release, as well as oncolysis of AB1 mesothelioma cells. MVTT elicits tumor-reactive CTLs, which are essential for curing malignant mesothelioma. MVTT virotherapy also induces chemotaxis that recruits IL-10-producing PMN-MDSCs into the TME, where they suppress DCs and therefore block the induction of antitumor CTLs. Depletion of PMN-MDSCs but not of M-MDSCs during MVTT virotherapy unleashes tumor-reactive CTLs leading to the therapeutic cure of a cancer, such as mesothelioma. The invention provides that the depletion of MDSCs, particularly PMN-MDSCs, in combination with oncolytic MVTT treatment, can restore potent antitumor T cell immunity, for example, by eliciting cytotoxic CD8⁺ T cell responses.

Accordingly, specific embodiments of the invention provide a method of treating a cancer, such as malignant mesothelioma or melanoma, by administering a combination of an oncolytic MVTT and a therapy that induces depletion of tumor-induced PMN-MDSCs.

Certain examples of therapies that induce depletion of tumor-induced PMN-MDSCs include gemcitabine, fluorouracil, bindarit, PDE5 inhibitors, tadalafil, nitroaspirin, COX-2 inhibitors, ipilimumab, bevacizumab, celecoxib, sildenafil and tadalafil, N-hydroxy-L-arginine, N-acetyl cysteine (NAC), CpG oligodeoxy-nucleotides (ODN), Bardoxolone methyl (CDDO-Me), withaferin A, Monoclonal anti-Gr1 antibody, IL4Ra aptamer, and peptibodies that target MDSC-membrane proteins (S100 family).

In specific embodiments, therapies that induce depletion of tumor-induced PMN-MDSCs are specific only for inducing depletion of tumor-induced PMN-MDSCs without affecting tumor-induced M-MDSCs.

In preferred embodiments, a therapy that induces depletion of tumor-induced PMN-MDSCs is an antibody against lymphocyte antigen 6 complex locus G6D (Ly6G), for example, antibody 1A8. An antibody against Ly6G, such as 1A8 specifically induces depletion of tumor-induced PMN-MDSCs without affecting tumor-induced M-MDSCs.

In certain embodiments, the methods comprise administering a chemotherapeutic agent before, during, or after administering a combination of an oncolytic virus and a therapy that induces depletion of tumor-induced PMN-MDSCs.

In further embodiments, an irradiation therapy is administered to the subject before or after administering a combination of an oncolytic virus and a therapy that induces depletion of tumor-induced PMN-MDSCs. An irradiation therapy can also be administered between administering an oncolytic virus and administering a therapy that induces depletion of tumor-induced PMN-MDSCs.

In certain embodiments, the methods comprise administering a check-point inhibitor to the subject before, during, or after administering a combination of an oncolytic virus and a therapy that induces depletion of tumor-induced PMN-MDSCs. A check-point inhibitor therapy can be administered between administering an oncolytic virus and administering a therapy that induces depletion of tumor-induced PMN-MDSCs.

Certain checkpoint inhibitors have been used in cancer therapy. Checkpoints refer to inhibitory pathways in the immune system that are responsible for maintaining self-tolerance and modulating the degree of immune system response to minimize peripheral tissue damage. Tumor cells can activate immune system checkpoints to decrease the efficacy of immune response against tumor tissues. Administering checkpoint inhibitors release the inhibition on the immune system and allow immune system activity against the tumor cells. Exemplary checkpoint inhibitors include inhibitors, such as antibodies, against cytotoxic T-lymphocyte antigen 4 (CTLA4, also known as CD152), programmed cell death protein 1 (PD-1, also known as CD279) and programmed cell death 1 ligand 1 (PD-L1, also known as CD274). Exemplary anti-PD-1 antibodies are commercially available and include pembrolizumab, lambrolizumab, nivolumab, AMP-224 (MERCK), and pidilizumab. Exemplary anti-PD-L1 antibodies are also commercially available and include atezolizumab, MDX-1105 (MEDAREX), MEDI4736 (MEDIMMUNE) MPDL3280A (GENENTECH), BMS-936559 (BRISTOL-MYERS SQUIBB), and AFFYMETRIX EBIOSCIENCE (MIH1). Exemplary anti-CTLA4 antibodies include ipilimumab (Bristol-Myers Squibb) and tremelimumab (PFIZER). Ipilimumab has recently received FDA approval for treatment of metastatic melanoma (Wada et al., 2013, J Transl Med 11:89). Additional checkpoint inhibitors are well known to a skilled artisan and such embodiments are within the purview of the invention.

Examples of cancers that can be treated according to the materials and methods disclosed herein include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer. In some embodiments, the cancer is melanoma, MDS, ovarian cancer, breast cancer, or multiple myeloma.

In some embodiments, the cancer is malignant mesothelioma or melanoma.

Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas. Examples of cancer types that may be treated with the compositions and methods of the invention are listed in Table 1.

TABLE 1
Examples of Cancer Types

| | |
|---|---|
| Acute Lymphoblastic Leukemia, Adult | Hairy Cell Leukemia |
| Acute Lymphoblastic Leukemia, | Head and Neck Cancer |
| Childhood | Hepatocellular (Liver) Cancer, Adult (Primary) |
| Acute Myeloid Leukemia, Adult | Hepatocellular (Liver) Cancer, Childhood |
| Acute Myeloid Leukemia, Childhood | (Primary) |
| Adrenocortical Carcinoma | Hodgkin's Lymphoma, Adult |
| Adrenocortical Carcinoma, Childhood | Hodgkin's Lymphoma, Childhood |
| AIDS-Related Cancers | Hodgkin's Lymphoma During Pregnancy |
| AIDS-Related Lymphoma | Hypopharyngeal Cancer |
| Anal Cancer | Hypothalamic and Visual Pathway Glioma, |
| Astrocytoma, Childhood Cerebellar | Childhood |
| Astrocytoma, Childhood Cerebral | Intraocular Melanoma |
| Basal Cell Carcinoma | Islet Cell Carcinoma (Endocrine Pancreas) |
| Bile Duct Cancer, Extrahepatic | Kaposi's Sarcoma |
| Bladder Cancer | Kidney (Renal Cell) Cancer |
| Bladder Cancer, Childhood | Kidney Cancer, Childhood |
| Bone Cancer, Osteosarcoma/Malignant | Laryngeal Cancer |
| Fibrous Histiocytoma | Laryngeal Cancer, Childhood |
| Brain Stem Glioma, Childhood | Leukemia, Acute Lymphoblastic, Adult |
| Brain Tumor, Adult | Leukemia, Acute Lymphoblastic, Childhood |
| Brain Tumor, Brain Stem Glioma, | Leukemia, Acute Myeloid, Adult |
| Childhood | Leukemia, Acute Myeloid, Childhood |
| Brain Tumor, Cerebellar Astrocytoma, | Leukemia, Chronic Lymphocytic |
| Childhood | Leukemia, Chronic Myelogenous |
| Brain Tumor, Cerebral | Leukemia, Hairy Cell |
| Astrocytoma/Malignant Glioma, | Lip and Oral Cavity Cancer |
| Childhood | Liver Cancer, Adult (Primary) |
| Brain Tumor, Ependymoma, Childhood | Liver Cancer, Childhood (Primary) |
| Brain Tumor, Medulloblastoma, | Lung Cancer, Non-Small Cell |
| Childhood | Lung Cancer, Small Cell |
| Brain Tumor, Supratentorial Primitive | Lymphoma, AIDS-Related |
| Neuroectodermal Tumors, Childhood | Lymphoma, Burkitt's |
| Brain Tumor, Visual Pathway and | Lymphoma, Cutaneous T-Cell, see Mycosis |
| Hypothalamic Glioma, Childhood | Fungoides and Sézary Syndrome |
| Brain Tumor, Childhood | Lymphoma, Hodgkin's, Adult |
| Breast Cancer | Lymphoma, Hodgkin's, Childhood |
| Breast Cancer, Childhood | Lymphoma, Hodgkin's During Pregnancy |
| Breast Cancer, Male | Lymphoma, Non-Hodgkin's, Adult |
| Bronchial Adenomas/Carcinoids, | Lymphoma, Non-Hodgkin's, Childhood |
| Childhood | Lymphoma, Non-Hodgkin's During Pregnancy |
| Burkitt's Lymphoma | Lymphoma, Primary Central Nervous System |
| Carcinoid Tumor, Childhood | Macroglobulinemia, Waldenström's |
| Carcinoid Tumor, Gastrointestinal | Malignant Fibrous Histiocytoma of |
| Carcinoma of Unknown Primary | Bone/Osteosarcoma |
| Central Nervous System Lymphoma, | Medulloblastoma, Childhood |
| Primary | Melanoma |
| Cerebellar Astrocytoma, Childhood | Melanoma, Intraocular (Eye) |
| Cerebral Astrocytoma/Malignant Glioma, | Merkel Cell Carcinoma |
| Childhood | Mesothelioma, Adult Malignant |
| Cervical Cancer | Mesothelioma, Childhood |
| Childhood Cancers | Metastatic Squamous Neck Cancer with Occult |
| Chronic Lymphocytic Leukemia | Primary |
| Chronic Myelogenous Leukemia | Multiple Endocrine Neoplasia Syndrome, |
| Chronic Myeloproliferative Disorders | Childhood |
| Colon Cancer | Multiple Myeloma/Plasma Cell Neoplasm |
| Colorectal Cancer, Childhood | Mycosis Fungoides |
| Cutaneous T-Cell Lymphoma, see | Myelodysplastic Syndromes |
| Mycosis Fungoides and Sézary | Myelodysplastic/Myeloproliferative Diseases |
| Syndrome | Myelogenous Leukemia, Chronic |
| Endometrial Cancer | Myeloid Leukemia, Adult Acute |
| Ependymoma, Childhood | Myeloid Leukemia, Childhood Acute |
| Esophageal Cancer | Myeloma, Multiple |
| Esophageal Cancer, Childhood | Myeloproliferative Disorders, Chronic |
| Ewing's Family of Tumors | Nasal Cavity and Paranasal Sinus Cancer |
| Extracranial Germ Cell Tumor, | Nasopharyngeal Cancer |
| Childhood | Nasopharyngeal Cancer, Childhood |
| Extragonadal Germ Cell Tumor | Neuroblastoma |
| Extrahepatic Bile Duct Cancer | Non-Hodgkin's Lymphoma, Adult |
| Eye Cancer, Intraocular Melanoma | Non-Hodgkin's Lymphoma, Childhood |
| Eye Cancer, Retinoblastoma | Non-Hodgkin's Lymphoma During Pregnancy |
| Gallbladder Cancer | Non-Small Cell Lung Cancer |
| Gastric (Stomach) Cancer | Oral Cancer, Childhood |
| Gastric (Stomach) Cancer, Childhood | Oral Cavity Cancer, Lip and |
| Gastrointestinal Carcinoid Tumor | Oropharyngeal Cancer |
| Germ Cell Tumor, Extracranial, | Osteosarcoma/Malignant Fibrous Histiocytoma |
| Childhood | of Bone |
| Germ Cell Tumor, Extragonadal | Ovarian Cancer, Childhood |
| Germ Cell Tumor, Ovarian | Ovarian Epithelial Cancer |
| Gestational Trophoblastic Tumor | Ovarian Germ Cell Tumor |
| Glioma, Adult | Ovarian Low Malignant Potential Tumor |
| Glioma, Childhood Brain Stem | Pancreatic Cancer |
| Glioma, Childhood Cerebral | Pancreatic Cancer, Childhood |
| Astrocytoma | Pancreatic Cancer, Islet Cell |
| Glioma, Childhood Visual Pathway and | Paranasal Sinus and Nasal Cavity Cancer |
| Hypothalamic | Parathyroid Cancer |
| Skin Cancer (Melanoma) | Penile Cancer |
| Skin Carcinoma, Merkel Cell | Pheochromocytoma |
| Small Cell Lung Cancer | Pineoblastoma and Supratentorial Primitive |
| Small Intestine Cancer | Neuroectodermal Tumors, Childhood |
| Soft Tissue Sarcoma, Adult | Pituitary Tumor |
| Soft Tissue Sarcoma, Childhood | Plasma Cell Neoplasm/Multiple Myeloma |
| Squamous Cell Carcinoma, see Skin | Pleuropulmonary Blastoma |
| Cancer (non-Melanoma) | Pregnancy and Breast Cancer |
| Squamous Neck Cancer with Occult | Pregnancy and Hodgkin's Lymphoma |
| Primary, Metastatic | Pregnancy and Non-Hodgkin's Lymphoma |
| Stomach (Gastric) Cancer | Primary Central Nervous System Lymphoma |
| Stomach (Gastric) Cancer, Childhood | Prostate Cancer |
| Supratentorial Primitive | Rectal Cancer |
| Neuroectodermal Tumors, Childhood | Renal Cell (Kidney) Cancer |
| T-Cell Lymphoma, Cutaneous, see | Renal Cell (Kidney) Cancer, Childhood |
| Mycosis Fungoides and Sézary | Renal Pelvis and Ureter, Transitional Cell |
| Syndrome | Cancer |
| Testicular Cancer | Retinoblastoma |
| Thymoma, Childhood | Rhabdomyosarcoma, Childhood |
| Thymoma and Thymic Carcinoma | Salivary Gland Cancer |
| Thyroid Cancer | Salivary Gland Cancer, Childhood |
| Thyroid Cancer, Childhood | Sarcoma, Ewing's Family of Tumors |
| Transitional Cell Cancer of the Renal | Sarcoma, Kaposi's |
| Pelvis and Ureter | Sarcoma, Soft Tissue, Adult |
| Trophoblastic Tumor, Gestational | Sarcoma, Soft Tissue, Childhood |
| Unknown Primary Site, Carcinoma of, | Sarcoma, Uterine |
| Adult | Sezary Syndrome |
| Unknown Primary Site, Cancer of, | Skin Cancer (non-Melanoma) |
| Childhood | Skin Cancer, Childhood |
| Unusual Cancers of Childhood | |
| Ureter and Renal Pelvis, Transitional Cell | |
| Cancer | |
| Urethral Cancer | |
| Uterine Cancer, Endometrial | |
| Uterine Sarcoma | |
| Vaginal Cancer | |
| Visual Pathway and Hypothalamic | |
| Glioma, Childhood | |
| Vulvar Cancer | |
| Waldenström's Macroglobulinemia | |
| Wilms' Tumor | |

As used herein, the term “tumor” refers to all neoplastic cell growth and proliferation, whether malignant or benign, and all pre-cancerous and cancerous cells and tissues. For example, a particular cancer may be characterized by a solid mass tumor or non-solid tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography) or magnetic resonance imaging (MM), or by needle aspirations. The use of these latter techniques is more common in early detection. Molecular and phenotypic analysis of cancer cells within a tissue can usually be used to confirm if the cancer is endogenous to the tissue or if the lesion is due to metastasis from another site. Some tumors are unresectable (cannot be surgically removed due to, for example the number of metastatic foci or because it is in a surgical danger zone). The treatment and prognostic methods of the invention can be utilized for early, middle, or late stage disease, and acute or chronic disease.

### Compositions and Treatments

Various methods may be used to deliver to a subject an oncolytic virus and/or a therapy that induces depletion of tumor-induced PMN-MDSCs. The oncolytic virus and the therapy that induces depletion of tumor-induced PMN-MDSCs can both be administered via the same route. Alternatively, the oncolytic virus can be administered via one route and the therapy that induces depletion of tumor-induced PMN-MDSCs can be administered via a different route. In preferred embodiments, the oncolytic virus and the therapy that induces depletion of tumor-induced PMN-MDSCs are both administered i.t.

The oncolytic viruses and the therapy that induces depletion of tumor-induced PMN-MDSC can be administered in one or more pharmaceutical compositions. The pharmaceutical compositions can include various other components. Examples of acceptable components or adjuncts which can be employed used in the pharmaceutical compositions include antioxidants, free radical scavenging agents, peptides, growth factors, antibiotics, bacteriostatic agents, immunosuppressives, anticoagulants, buffering agents, anti-inflammatory agents, anti-angiogenics, anti-pyretics, time-release binders, anesthetics, steroids, and corticosteroids. Such components can provide additional therapeutic benefit, enhance the therapeutic action of the anti-cancer therapy or act towards preventing any potential side effects of the anti-cancer therapy.

Additional agents can be co-administered to subjects or into the cancer cells in a subject in the same or separate formulations. Such additional agents include agents that modify a given biological response, such as immunomodulators. The additional agents may be, for example, small molecules, polypeptides (proteins, peptides, or antibodies or antibody fragments), or nucleic acids (encoding polypeptides or inhibitory nucleic acids such as antisense oligonucleotides or interfering RNA). For example, proteins such as tumor necrosis factor (TNF), interferon (such as alpha-interferon and beta-interferon), nerve growth factor (NGF), platelet derived growth factor (PDGF), and tissue plasminogen activator can be administered. Biological response modifiers, such as lymphokines, interleukins (such as interleukin-1 (IL-1), interleukin-2 (IL-2), and interleukin-6 (IL-6)), granulocyte macrophage colony stimulating factor (GM-CSF), granulocyte colony stimulating factor (G-CSF), or other growth factors can be administered. In one embodiment, the methods and compositions of the invention incorporate one or more anti-cancer agents, such as cytotoxic agents, chemotherapeutic agents, anti-signaling agents, and anti-angiogenic agents.

In some embodiments, the compositions of the invention include at least one additional anti-cancer agent (e.g., a chemotherapeutic agent). In some embodiments of the methods of the invention, at least one additional anti-cancer agent is administered with the compositions of the invention. In some embodiments, the anti-cancer agent is selected from among suberoylanilide hydroxamic acid (SAHA) or other histone deacetylase inhibitor, arsenic trioxide, doxorubicin or other anthracycline DNA intercalating agent, and etoposide or other topoisomerase II inhibitor.

In some embodiments, the compositions can include, and the methods can include administering, one or more proteasome inhibitors (e.g., bortezomib), inhibitors of autophagy (e.g., chloroquine), alkylating agents (e.g., melphalan, cyclophosphamide), MEK inhibitors (e.g., PD98509), FAK/PYK2 inhibitors (e.g., PF562271), or EGFR inhibitors (e.g., erlotinib, gefitinib, cetuximab, panitumumab, zalutumumab, nimotuzumab, and matuzumab), or a combination of two or more of the foregoing.

Thus, an oncolytic virus or a therapy that induces depletion of tumor-induced PMN-MDSCs, whether administered separately, or as a pharmaceutical composition, can include various other components as additives. Examples of acceptable components or adjuncts which can be employed in relevant circumstances include antioxidants, free radical scavenging agents, peptides, growth factors, antibiotics, bacteriostatic agents, immunosuppressives, anticoagulants, buffering agents, anti-inflammatory agents, anti-angiogenics, anti-pyretics, time-release binders, anesthetics, steroids, and corticosteroids. Such components can provide additional therapeutic benefit, act to affect the therapeutic action of the compounds of the invention, or act towards preventing any potential side effects which may be posed as a result of administration of the compounds. The immunotherapeutic agent can be conjugated to a therapeutic agent or other agent, as well.

As used herein, the term “immunotherapy” refers to the treatment of disease via the stimulation, induction, subversion, mimicry, enhancement, augmentation or any other modulation of a subject's immune system to elicit or amplify adaptive or innate immunity (actively or passively) against cancerous or otherwise harmful proteins, cells or tissues. Immunotherapies (i.e., immunotherapeutic agents) include cancer vaccines, immunomodulators, monoclonal antibodies (e.g., humanized monoclonal antibodies), immunostimulants, dendritic cells, and viral therapies, whether designed to treat existing cancers or prevent the development of cancers or for use in the adjuvant setting to reduce likelihood of recurrence of cancer. Examples of cancer vaccines include GVAX, Stimuvax, DCVax and other vaccines designed to elicit immune responses to tumor and other antigens including MUC1, NY-ESO-1, MAGE, p53 and others. Examples of immunomodulators include 1MT, Ipilimumab, Tremelimumab and/or any drug designed to de-repress or otherwise modulate cytotoxic or other T cell activity against tumor or other antigens, including, but not restricted to, treatments that modulate T-Reg cell control pathways via CTLA-4, CD80, CD86, MHC, B7-DC, B7-H1, B7-H2, B7-H3, B7-H4, CD28, other TCRs, PD-1, PDL-1, CD80, ICOS and their ligands, whether via blockade, agonist or antagonist. Examples of immunostimulants include corticosteroids and any other anti- or pro-inflammatory agent, steroidal or non-steroidal, including, but not restricted to, GM-CSF, interleukins (e.g., IL-2, IL-7, IL-12), cytokines such as the interferons, and others. Examples of dendritic cell (DC) therapies include modified dendritic cells and any other antigen presenting cell, autologous, allogeneic, or xenogeneic, whether modified by multiple antigens, whole cancer cells, single antigens, by mRNA, phage display or any other modification, including but not restricted to ex vivo-generated, antigen-loaded dendritic cells (DCs) to induce antigen-specific T-cell immunity, ex vivo gene-loaded DCs to induce humoral immunity, ex vivo-generated antigen-loaded DCs induce tumor-specific immunity, ex vivo-generated immature DCs to induce tolerance, including but not limited to Provenge and others. Examples of viral therapies include oncolytic viruses or virus-derived genetic or other material designed to elicit anti-tumor immunity and inhibitors of infectious viruses associated with tumor development, such as drugs in the Prophage series. Examples of monoclonal antibodies include Alemtuzumab, Bevacizumab, Cetuximab, Gemtuzumab ozogamicin, Rituximab, Trastuzumab, Radioimmunotherapy, Ibritumomab tiuxetan, Tositumomab/iodine tositumomab regimen. An immunotherapy may be a monotherapy or used in combination with one or more other therapies (one or more other immunotherapies or non-immunotherapies).

As used herein, the term “cytotoxic agent” refers to a substance that inhibits or prevents the function of cells and/or causes destruction of cells in vitro and/or in vivo. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, toxins such as small molecule toxins or enzymatically active toxins of bacterial, fungal, plant or animal origin, and antibodies, including fragments and/or variants thereof.

As used herein, the term “chemotherapeutic agent” is a chemical compound useful in the treatment of cancer, such as, for example, taxanes, e.g., paclitaxel (TAXOL, BRISTOL-MYERS SQUIBB Oncology, Princeton, N.J.) and doxetaxel (TAXOTERE, Rhone-Poulenc Rorer, Antony, France), chlorambucil, vincristine, vinblastine, anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON, GTx, Memphis, Tenn.), and anti-androgens such as flutamide, nilutamide, bicalutamide, leuprolide, and goserelin, etc. Examples of anti-cancer agents, including chemotherapeutic agents that may be used in conjunction with the compounds of the invention are listed in Table 2. In a preferred embodiment, the chemotherapeutic agent is one or more anthracyclines. Anthracyclines are a family of chemotherapy drugs that are also antibiotics. The anthracyclines act to prevent cell division by disrupting the structure of the DNA and terminate its function by: (1) intercalating into the base pairs in the DNA minor grooves; and (2) causing free radical damage of the ribose in the DNA. The anthracyclines are frequently used in leukemia therapy. Examples of anthracyclines include daunorubicin (CERUBIDINE), doxorubicin (ADRIAMYCIN, RUBEX), epirubicin (ELLENCE, PHARMORUBICIN), and idarubicin (IDAMYCIN).

TABLE 2
Examples of Anti-Cancer Agents

| | |
|---|---|
| 13-cis-Retinoic Acid | Mylocel |
| 2-Amino-6- | Letrozole |
| Mercaptopurine | Neosar |
| 2-CdA | Neulasta |
| 2-Chlorodeoxyadenosine | Neumega |
| 5-fluorouracil | Neupogen |
| 5-FU | Nilandron |
| 6 - TG | Nilutamide |
| 6 - Thioguanine | Nitrogen Mustard |
| 6-Mercaptopurine | Novaldex |
| 6-MP | Novantrone |
| Accutane | Octreotide |
| Actinomycin-D | Octreotide acetate |
| Adriamycin | Oncospar |
| Adrucil | Oncovin |
| Agrylin | Ontak |
| Ala-Cort | Onxal |
| Aldesleukin | Oprevelkin |
| Alemtuzumab | Orapred |
| Alitretinoin | Orasone |
| Alkaban-AQ | Oxaliplatin |
| Alkeran | Paclitaxel |
| All-transretinoic acid | Pamidronate |
| Alpha interferon | Panretin |
| Altretamine | Paraplatin |
| Amethopterin | Pediapred |
| Amifostine | PEG Interferon |
| Aminoglutethimide | Pegaspargase |
| Anagrelide | Pegfilgrastim |
| Anandron | PEG-INTRON |
| Anastrozole | PEG-L-asparaginase |
| Arabinosylcytosine | Phenylalanine Mustard |
| Ara-C | Platinol |
| Aranesp | Platinol-AQ |
| Aredia | Prednisolone |
| Arimidex | Prednisone |
| Aromasin | Prelone |
| Arsenic trioxide | Procarbazine |
| Asparaginase | PROCRIT |
| ATRA | Proleukin |
| Avastin | Prolifeprospan 20 with Carmustine implant |
| BCG | Purinethol |
| BCNU | Raloxifene |
| Bevacizumab | Rheumatrex |
| Bexarotene | Rituxan |
| Bicalutamide | Rituximab |
| BiCNU | Roveron-A (interferon alfa-2a) |
| Blenoxane | Rubex |
| Bleomycin | Rubidomycin hydrochloride |
| Bortezomib | Sandostatin |
| Busulfan | Sandostatin LAR |
| Busulfex | Sargramostim |
| C225 | Solu-Cortef |
| Calcium Leucovorin | Solu-Medrol |
| Campath | STI-571 |
| Camptosar | Streptozocin |
| Camptothecin-11 | Tamoxifen |
| Capecitabine | Targretin |
| Carac | Taxol |
| Carboplatin | Taxotere |
| Carmustine | Temodar |
| Carmustine wafer | Temozolomide |
| Casodex | Teniposide |
| CCNU | TESPA |
| CDDP | Thalidomide |
| CeeNU | Thalomid |
| Cerubidine | TheraCys |
| cetuximab | Thioguanine |
| Chlorambucil | Thioguanine Tabloid |
| Cisplatin | Thiophosphoamide |
| Citrovorum Factor | Thioplex |
| Cladribine | Thiotepa |
| Cortisone | TICE |
| Cosmegen | Toposar |
| CPT-11 | Topotecan |
| Cyclophosphamide | Toremifene |
| Cytadren | Trastuzumab |
| Cytarabine | Tretinoin |
| Cytarabine liposomal | Trexall |
| Cytosar-U | Trisenox |
| Cytoxan | TSPA |
| Dacarbazine | VCR |
| Dactinomycin | Velban |
| Darbepoetin alfa | Velcade |
| Daunomycin | VePesid |
| Daunorubicin | Vesanoid |
| Daunorubicin | Viadur |
| hydrochloride | Vinblastine |
| Daunorubicin liposomal | Vinblastine Sulfate |
| DaunoXome | Vincasar Pfs |
| Decadron | Vincristine |
| Delta-Cortef | Vinorelbine |
| Deltasone | Vinorelbine tartrate |
| Denileukin diftitox | VLB |
| DepoCyt | VP-16 |
| Dexamethasone | Vumon |
| Dexamethasone acetate | Xeloda |
| dexamethasone sodium | Zanosar |
| phosphate | Zevalin |
| Dexasone | Zinecard |
| Dexrazoxane | Zoladex |
| DHAD | Zoledronic acid |
| DIC | Zometa |
| Diodex | Gliadel wafer |
| Docetaxel | Glivec |
| Doxil | GM-CSF |
| Doxorubicin | Goserelin |
| Doxorubicin liposomal | granulocyte - colony stimulating factor |
| Droxia | Granulocyte macrophage colony stimulating |
| DTIC | factor |
| DTIC-Dome | Halotestin |
| Duralone | Herceptin |
| Efudex | Hexadrol |
| Eligard | Hexalen |
| Ellence | Hexamethylmelamine |
| Eloxatin | HMM |
| Elspar | Hycamtin |
| Emcyt | Hydrea |
| Epirubicin | Hydrocort Acetate |
| Epoetin alfa | Hydrocortisone |
| Erbitux | Hydrocortisone sodium phosphate |
| Erwinia L-asparaginase | Hydrocortisone sodium succinate |
| Estramustine | Hydrocortone phosphate |
| Ethyol | Hydroxyurea |
| Etopophos | Ibritumomab |
| Etoposide | Ibritumomab Tiuxetan |
| Etoposide phosphate | Idamycin |
| Eulexin | Idarubicin |
| Evista | Ifex |
| Exemestane | IFN-alpha |
| Fareston | Ifosfamide |
| Faslodex | IL - 2 |
| Femara | IL-11 |
| Filgrastim | Imatinib mesylate |
| Floxuridine | Imidazole Carboxamide |
| Fludara | Interferon alfa |
| Fludarabine | Interferon Alfa-2b (PEG conjugate) |
| Fluoroplex | Interleukin - 2 |
| Fluorouracil | Interleukin-11 |
| Fluorouracil (cream) | Intron A (interferon alfa-2b) |
| Fluoxymesterone | Leucovorin |
| Flutamide | Leukeran |
| Folinic Acid | Leukine |
| FUDR | Leuprolide |
| Fulvestrant | Leurocristine |
| G-CSF | Leustatin |
| Gefitinib | Liposomal Ara-C |
| Gemcitabine | Liquid Pred |
| Gemtuzumab ozogamicin | Lomustine |
| Gemzar | L-PAM |
| Gleevec | L-Sarcolysin |
| Lupron | Meticorten |
| Lupron Depot | Mitomycin |
| Matulane | Mitomycin-C |
| Maxidex | Mitoxantrone |
| Mechlorethamine | M-Prednisol |
| Mechlorethamine | MTC |
| Hydrochlorine | MTX |
| Medralone | Mustargen |
| Medrol | Mustine |
| Megace | Mutamycin |
| Megestrol | Myleran |
| Megestrol Acetate | Iressa |
| Melphalan | Irinotecan |
| Mercaptopurine | Isotretinoin |
| Mesna | Kidrolase |
| Mesnex | Lanacort |
| Methotrexate | L-asparaginase |
| Methotrexate Sodium | LCR |
| Methylprednisolone | |

While oncolytic viruses and/or the therapies that induce depletion of tumor-induced PMN-MDSC of the invention can be administered to subjects as isolated agents, it is preferred to administer these viruses or therapies as part of a pharmaceutical composition. Therefore, the subject invention thus further provides compositions comprising a combination of an oncolytic virus, a compound that induces depletion of tumor-induced PMN-MDSC, and at least one pharmaceutically acceptable carrier. The pharmaceutical compositions can be adapted for various routes of administration, such as enteral, parenteral, intravenous, intramuscular, topical, subcutaneous, and so forth. Administration can be continuous or at distinct intervals, as can be determined by a person of ordinary skill in the art. A “pharmaceutically acceptable carrier” refers to an ingredient in a pharmaceutical formulation, other than an active ingredient, and includes, but is not limited to, a buffer, excipient, stabilizer, or preservative.

The compositions administered in accordance with the methods of the invention can be formulated according to known methods for preparing pharmaceutically useful compositions. Formulations are described in a number of sources which are well known and readily available to those skilled in the art. For example, Remington's Pharmaceutical Science (Martin, E. W., 1995, Easton Pa., Mack Publishing Company, 19^{th} ed.) describes formulations which can be used in connection with the subject invention. Formulations suitable for administration include, for example, aqueous sterile injection solutions, which may contain antioxidants, buffers, bacteriostats, and solutes that render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the compositions of the subject invention can include other agents conventional in the art having regard to the type of formulation in question.

Compositions of the invention, the oncolytic viruses, the therapies that induce depletion of tumor-induced PMN-MDSC, and others agents used in the methods of the invention may be locally administered at one or more anatomical sites, such as sites of unwanted cell growth (such as a tumor site, e.g., injected or topically applied to the tumor), optionally in combination with a pharmaceutically acceptable carrier such as an inert diluent. Compositions of the invention and other agents used in the methods of the invention may be systemically administered, such as intravenously or orally, optionally in combination with a pharmaceutically acceptable carrier such as an inert diluent, or an assimilable edible carrier for oral delivery. They may be enclosed in hard or soft shell gelatin capsules, may be compressed into tablets, or may be incorporated directly with the food of the patient's diet. For oral therapeutic administration, the agents may be combined with one or more excipients and used in the form of ingestible tablets, buccal tablets, troches, capsules, elixirs, suspensions, syrups, wafers, aerosol sprays, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the compositions and agents may be incorporated into sustained-release preparations and devices.

The oncolytic viruses and/or the therapies that induce depletion of tumor-induced PMN-MDSC can be administered into the tumor (intra-tumorally) or into a lymph node, such as inguinal lymph node of the subject. The oncolytic viruses and/or the therapies that induce depletion of tumor-induced PMN-MDSC can also be administered intradermally, intravenously, or intraperitoneally by infusion or injection.

Solutions of the active agents can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating the oncolytic viruses and/or the therapies that induce depletion of tumor-induced PMN-MDSC in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compositions and agents may be applied in pure-form, i.e., when they are liquids. However, it will generally be desirable to administer them topically to the skin as compositions, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the peptide can be dissolved or dispersed at effective levels, optionally with the aid of non-toxic surfactants. Additives such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver the peptides to the skin are disclosed in Jacquet et al. (U.S. Pat. No. 4,608,392), Geria (U.S. Pat. No. 4,992,478), Smith et al. (U.S. Pat. No. 4,559,157) and Woltzman (U.S. Pat. No. 4,820,508).

Useful dosages of the pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Accordingly, the present invention includes a pharmaceutical composition comprising the oncolytic viruses and/or the therapies that induce depletion of tumor-induced PMN-MDSC, optionally, in combination with a pharmaceutically acceptable carrier. Pharmaceutical compositions adapted for oral, topical or parenteral administration, comprising an oncolytic virus and/or a therapy that induces depletion of tumor-induced PMN-MDSCs constitute a preferred embodiment of the invention. The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition. Advantageously, in some embodiments, administration of the compounds of the invention does not induce weight loss or overt signs of toxicity in the subject.

A suitable dose(s) results in a concentration of the active agent in cancer tissue, such as a malignant tumor, which is known to achieve the desired response. The preferred dosage is the amount which results in maximum inhibition of cancer cell growth, without unmanageable side effects. Administration of the oncolytic viruses and the therapies that induce depletion of tumor-induced PMN-MDSC and optionally, other agents can be continuous or at distinct intervals.

To provide for the administration of such dosages for the desired therapeutic treatment, in some embodiments, pharmaceutical compositions of the invention can comprise between about 0.1% and 45%, and especially, 1 and 15%, by weight of the total of one or more of the agents of the invention based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

### Definitions

To facilitate the understanding of the subject matter disclosed herein, a number of terms, abbreviations or other shorthand as used herein are defined below. Any term, abbreviation or shorthand not defined is understood to have the ordinary meaning used by a skilled artisan contemporaneous with the submission of this application.

The term “subject,” as used herein, describes a mammal including, but not limited to, humans, apes, chimpanzees, orangutans, monkeys, dogs, cats, horses, pigs, sheep, goats, mice, rats, and guinea pigs.

The term “treatment” or any grammatical variation thereof (e.g., treat, treating, and treatment etc.), as used herein, includes, but is not limited to, ameliorating or alleviating a symptom of a disease or condition; reducing or delaying recurrence of a condition; reducing, suppressing, inhibiting, lessening, or affecting the progression and/or severity of an undesired physiological change or a diseased condition. For instance, treatment includes, for example, preventing, inhibiting, or slowing the rate of development of a cancer or conversion of a benign cancer into a malignant cancer; slowing the growth and/or proliferation of cancer; and reducing the size or spread of cancer.

The term “effective amount,” as used herein, refers to an amount that is capable of treating or ameliorating a cancer or is otherwise capable of producing an intended therapeutic effect. In certain embodiments, the effective amount enables a 5%, 10%, 20%, 30%, 40%, 50%, 75%, 90%, 95%, 99% or 100% reduction in the rate of formation of a tumor or spread of a cancer. In certain embodiments, the effective amount enables a 5%, 10%, 15%, 20%, 25%, 30%, 35%, or 40% reduction in the size of a tumor or the spread of a cancer.

As used herein, the singular forms “a,” “an” and “the” are intended to include the plural forms as well, unless the context clearly indicates otherwise. Thus, for example, a reference to “a compound” includes more than one such compound. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” “consisting essentially of,” “consists essentially of,” “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

With respect to an oncolytic virus having a deletion of a gene, the term “deletion” refers to genetic modifications done to the gene including any of the open reading frame, upstream regulatory region and downstream regulatory region that result in down regulation or complete inhibition of the transcription of the open reading frame (ORF) of the gene. Deletion can be achieved either by deleting the entire ORF or a portion of the ORF, for example, by introducing: a frame shift mutation, a missense mutation, a sequence that disrupt the activity of the protein encoded by the gene, a stop codon, or any combination thereof.

With respect to a virus containing a heterologous gene, the term “heterologous gene” includes an open reading frame and can further optionally comprise one or more additional elements of a gene, such as an upstream regulatory region, a downstream regulatory region, and/or a terminator.

### Materials and Methods

### Mice

All mice were maintained according to approved procedures. 6-8 week-old female BALB/c and C57BL/6N mice were used.

### Cell Culture

Vero cells, purchased from ATCC, and B16F10 cells, a kind gift, were maintained in complete Dulbecco's modified Eagle's medium (DMEM, Gibco; supplemented with 10% FBS and antibiotics). AB1 cell line, purchased from European Collection of Cell Cultures, was maintained in complete Roswell Park Memorial Institute-1640 medium (RPMI, Gibco; supplemented with 10% FBS, 2 mM L-glutamine and antibiotics). Luciferase-expressing cells were maintained in complete RPMI supplemented with 1 μg/ml puromycin (Invitrogen). T cells and splenocytes were cultured in complete RPMI supplemented with 50 μM 2-mercaptoethanol (Sigma).

### Virus and In Vitro Infection

A highly attenuated MVTT virus encoding dual reporters of HcRed and HIV-1 p24 was prepared. MVTT viral stocks were prepared and virus titers were determined by plaque forming assay in Vero cells using serially diluted virus. In vitro infection was performed in 24-well plate with 2×10⁵ AB1 mesothelioma cells in each well. 0.2 MOI recombinant MVTT was added into the culture to allow 1 hour attachment before cells were washed and incubated with 1 ml fresh medium. Culture supernatants were harvested 24, 48, and 72 hours after infection, and viral titers were measured by serial dilution and plaque forming assay in Vero cells. Released HMGB1 were examined by western blotting using anti-HMGB1 antibody (Abcam, ab79823). Released ATP in the supernatant and cell viability were determined by CellTiter-Glo luminescent cell viability assay (Promega) per the manufacturer's instructions. Relative cell viability was calculated with ratio of luminescence between infected cells and uninfected cells. Cells were also detached and incubated with anti-CRT antibody (Abcam, ab92516) for surface labelling and flow cytometric analysis. CRT expression in the cell lysates was also determined by western blotting. AB1-MVTT viral supernatant used for antigen-presentation assay was collected 48 hours after infection. Cell debris was removed by centrifugation, passed through a 0.2 μm low-protein binding membrane (Millipore) and heat-inactivated at 60° C. for 1 hour. Successful elimination of live virus was confirmed by plaque forming assay in Vero cells.

### Tumor Models and Intra-Tumoral Treatment

Mesothelioma AB1 cells or melanoma B16F10 cells were harvested and single cell suspensions of 5×10⁵ cells in 100 μl PBS were injected s.c. into right hind flank of BALB/c or C57BL/6N mice, respectively. Tumor volumes were measured by caliper and calculated with the formula: Tumor volume=1/2(length×width). Luciferase-expressing tumors were also measured by bioluminescence imaging using an IVIS spectrum (PerkinElmer) and signal intensity was presented as photons/s/cm²/sr within regions of interest (ROI) using Living Image software (version 4.0, PerkinElmer), as previously described. Intra-tumoral treatment of established tumors was started at 7 days after tumor inoculation. Tumors were injected with 100 μl of recombinant MVTT, anti-Ly6G antibody (clone 1A8, BioXCell) or combination of the two. 1A8 was administered at 100 μg per dose and rat IgG2a (clone 2A3, BioXcell) was injected alone or in combination with recombinant MVTT as an isotype control. Mice that rejected tumors were re-challenged with 2×10⁶ tumor cells via an s.c. injection on their opposite flank. All animals were euthanized when tumor length reached more than 15 mm.

### Ex Vivo Cell Preparation

Splenocytes were isolated as previously described. Tumors were cut into pieces and digested with 1 mg/ml collagenase IV (Sigma) and 0.5 U/ml Dnase I (Roche) for 1.5 hours at 37° C. Cells were passed through a 70 μm strainer and then subjected to 40%/80% Percoll gradient (Sigma). Leukocytes at the interphase were recovered after centrifuge at 800 g for 20 min. Bone-marrow leukocytes were flushed out from tibia and femur. Cells were then passed through a 70 μm strainer and red blood cells were removed using red blood lysis buffer (BD Biosciences).

### T Cells and MDSCs Isolation

Single-cell suspensions of splenocytes were used for cell isolation. CD3⁺ T cells were isolated using Dynabeads Untouched T Cell Kits (Thermo Scientific). CD4⁺ and CD8⁺ T cells were isolated using T Cell Isolation Kit (Miltenyi). Total MDSCs or MDSCs subsets were isolated using MDSCs Isolation Kit (Miltenyi), according to manufacturer's instructions.

### Adoptive MDSCs Transfer

Purified MDSCs were labelled with CFSE (Thermo Scientific). 4×10⁶ MDSCs were intravenously injected into AB1 tumor-bearing mice through tail vein. Labelled MDSCs were detected 24 hours after transfer.

### In Vivo Cell Depletion

CD4⁺ and CD8⁺ T cells were depleted during treatment by intraperitoneal injection of 250 μg anti-CD4 (YTS191.1, BioXcell) or anti-CD8 (YTS169.4, BioXcell), respectively, every 5 days, starting 1 day before therapy. Successful T cell depletion was confirmed by flow cytometric analysis of peripheral blood mononuclear cell (PBMC). Anti-Ly6G (clone 1A8) and corresponding isotype (clone 2A3) were also purchased from BioXcell.

### Measurement of Cytokine and Chemokine Production

Cytokine concentrations in the culture supernatant were measured by LEGENDplex T Helper Cytokine Panel (BioLegend). Tumors were cut into pieces and homogenized in T-PER Tissue Protein Extraction Reagent (Thermo Scientific) supplemented with Protease Inhibitor Cocktail (Roche). Chemokine concentrations were determined by LEGENDplex Proinflammatory Chemokine Panel (BioLegend) and normalized against total proteins determined by BCA protein assay (Thermo Scientific).

### BMDCs Culture, In Vitro Antigen-Presentation and Suppression Assays

Following a standard protocol, isolated bone-marrow cells were plated in 6-well plate at 3×10⁶ cell per well in the presence of 40 ng/ml GM-CSF and IL-4. Half of the differentiation medium was replaced every 2 days. On day 9, loosely adherent cells were resuspended by repeated pipetting and collected together with non-adherent cells in the supernatant for flow cytometric analysis with surface staining of anti-CD3, anti-CD11c and anti-WIC II, resulting in >90% CD11c⁺MHC II⁺ BMDCs. For BMDCs-T cells co-culture, BMDCs were pooled and seeded into 96-well V-bottom plate at 2×10⁴ cells per well in the presence of 100 μl inactivated AB1-MVTT viral supernatant or culture medium. In some cultures, anti-CRT antibody (Abcam, ab92516) or rabbit IgG was added at 100 ng/ml. After incubation overnight, BMDCs were thoroughly washed with culture medium and CFSE labelled CD3⁺ T cells were added at a ratio of 1:1, for an additional culture of 10 days, with replacement of half of the culture medium every 4 days. Culture supernatant collected on day 7 and cells collected on day 10 were subjected to analysis of cytokine secretion and T cell proliferation, respectively. For BMDCs-MDSCs co-culture, BMDCs were seeded in 96-well U-bottom plate at 5×10⁴ cells per well, stimulated by 100 ng/ml LPS (Sigma) or 100 μl inactivated AB1-MVTT viral supernatant, in the presence of purified PMN-MDSCs or M-MDSCs. To clearly distinguish BMDCs from MDSCs by flow cytometry, purified MDSCs subsets were labelled with CFSE prior to incubation with BMDCs. 48 hours after LPS-stimulation, BMDCs maturation was assessed via flow cytometry. When cells were stimulated with AB1-MVTT viral supernatant, half of the medium was replaced with fresh culture medium on day 4 and supernatant was collected on day 7 to assess cytokine secretion.

### IL-10 Receptor Blocking Assay

BMDCs were seeded in 96-well U-bottom plate at 5×10⁴ cells per well and were subjected to incubate with 5 μg/ml anti-mouse CD210 (IL-10R, clone 1B1.3a, BioLegend) antibody for 30 min at 37° C. Then 1×10⁵ CFSE labelled PMN-MDSCs or M-MDSCs were added into the culture at a ratio of 2:1 with BMDCs, following stimulation with 100 ng/ml LPS for 48 hours in the incubator. Culture volume was maintained at 100 p1 each well and rat IgG1 (eBioscience) was used as isotype control.

### Flow Cytometry

Cell surface and intracellular immunostaining were performed as previously described. The following antibodies were purchased from eBioscience: anti-CD11b (clone M1/70), anti-Ly6C (clone HK1.4), anti-Ly6G (clone 1A8-Ly6 g), anti-CD3 (clone 17A2), anti-CD4 (clone GK1.5), anti-CD8 (clone 53-6.7), anti-PD1 (clone J43), anti-Tim3 (clone RMT3-23), anti-CD11c (clone N418), anti-MHC II (clone M5/114.15.2), anti-CD80 (clone 16-10A1), and anti-CD49b (clone DX5). The following antibodies were purchased from BioLegend: anti-CD25 (clone 3C7), anti-Foxp3 (clone 150D), anti-CXCR2 (clone SA045E1), and anti-CXCR3 (clone CXCR3-173). Anti-CCR2 (clone REA538) antibody was purchased from Miltenyi. Samples were run on a BD FACSAria II cell sorter (BD Biosciences) and analyzed using FlowJo (Tree Star, v10).

### ELISpot and T Cell Cytotoxicity Assay

IFN-γ-producing T cells in isolated splenocytes were assessed by ELISpot assay. gp70-AH1 (SPSYVYHQF), OVA257-264(SIINFEKL), GP100 (EGPRNQDWL), TRP2 (SVYDFFVWL), and TWIST1 peptides (15-mers spanning the entire amino acid sequence with 11 amino acids overlapping) were synthesized by GL Biochem (Shanghai). Cytotoxic effect of purified T cells against AB1 cells was determined using LIVE/DEAD Viability/Cytotoxicity Kit (Thermo Scientific), as previously described.

### Statistical Analyses

All data are presented as mean±s.e.m. Significance was determined by the two-tailed Student t-test and p-value <0.05 was considered statistically significant. Survival of all animals was plotted on Kaplan-Meier survival curve and the log-rank test was performed to analyze differences in GraphPad Prism 5 software.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Oncolysis of Mesothelioma Cells by MVTT Triggers Exposure of CRT as Well as Release of HMGB1 and ATP

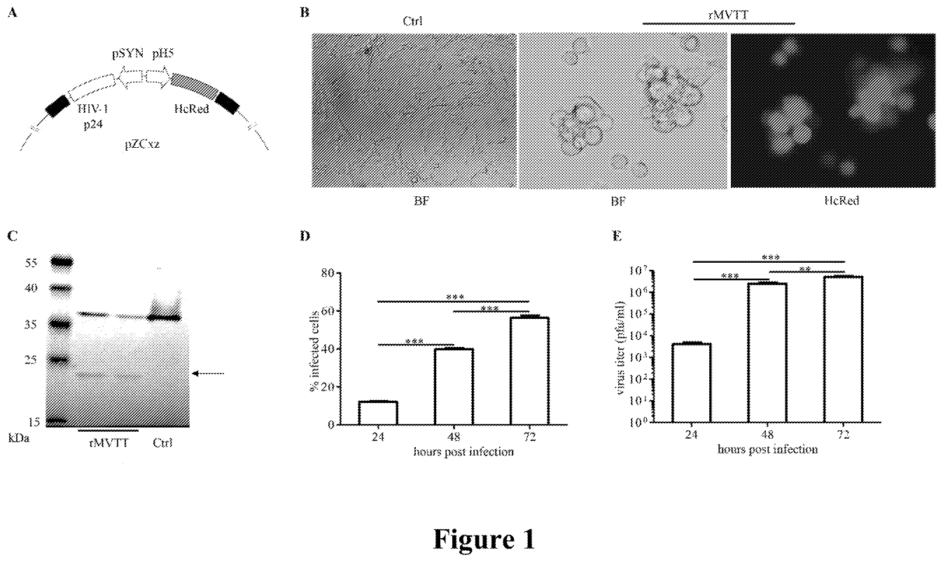

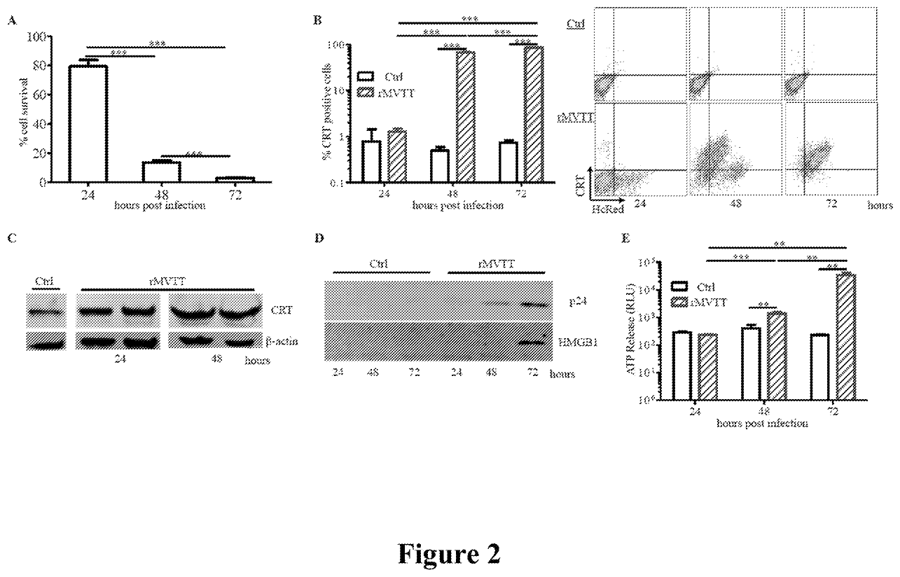

To determine the oncolytic effects of MVTT, a recombinant MVTT (rMVTT) was generated to simultaneously express two detection markers, HIV-1 p24 and far-red fluorescent mutant HcRed (FIG. 1A). Expression of two makers facilitates the detection of viral replication as well as encoded gene expression. MVTT has a broad range for mammalian cell infection. AB1 mesothelioma cells were susceptible to the rMVTT infection, displaying the presence of red fluorescent syncytia (FIG. 1B) and expression of virus-encoded p24 protein (FIG. 1C). An increase of the HcRed signal and released free virus overtime indicated that the rMVTT virus can infect and replicate in AB1 cells (FIGS. 1D-1E). The oncolytic ability of rMVTT was subsequently determined, showing that the viral infection significantly decreased AB1 cell viability (FIG. 2A). Calreticulin (CRT), a DAMP that is typically in the lumen of the endoplasmic reticulum, is translocated after the induction of immunogenic apoptosis to the surface of dying cells, at which it functions as an eat-me signal for professional phagocytes. Therefore, the expression of CRT protein in AB1 cells was determined after MVTT infection by flow cytometric analysis. When using 0.2 MOI rMVTT for infection, less than 5% of AB1 cells showed exposure of CRT on their surface after 24 hours. Due to active viral replication, however, the percentage increased to 70% and 90% at 48 and 72 hours post infection, respectively (FIG. 2B, left panel). Importantly, all the CRT positive cells were showing expression of HcRed, suggesting that rMVTT infection was the cause of the exposed CRT protein (FIG. 2B, right panel). Furthermore, Western blot analysis also demonstrated that rMVTT infection caused the upregulated expression of CRT protein in AB1 cells (FIG. 2C). Besides CRT protein, release of other DAMPs such as high mobility group box 1 (HMGB1) and ATP from dying cells may activate antigen-presenting cells (APCs) to mount antitumor immunity. Therefore, expression of CRT and HMGB1 proteins was measured to test the possibility that oncolysis might lead to immunogenic cell death. HMGB1 protein could be readily detected in the culture supernatant 72 hours post rMVTT infection but not in uninfected AB1 cell control (FIG. 2D). Moreover, the released ATP in the supernatant was also significantly increased after rMVTT infection overtime (FIG. 2E). Thus, oncolysis of AB1 mesothelioma cells by rMVTT induced the upregulated expression and exposure of CRT as well as release of ATP and HMGB1 from dying cells, which are commonly recognized as the three major hallmarks of immunogenic cell death for provoking adaptive antitumor immune responses.

### Example 2—rMVTT Treatment Eliminated Established AB1 Tumors Dose-Dependently Yet Failed to Mount Antitumor T Cell Immunity

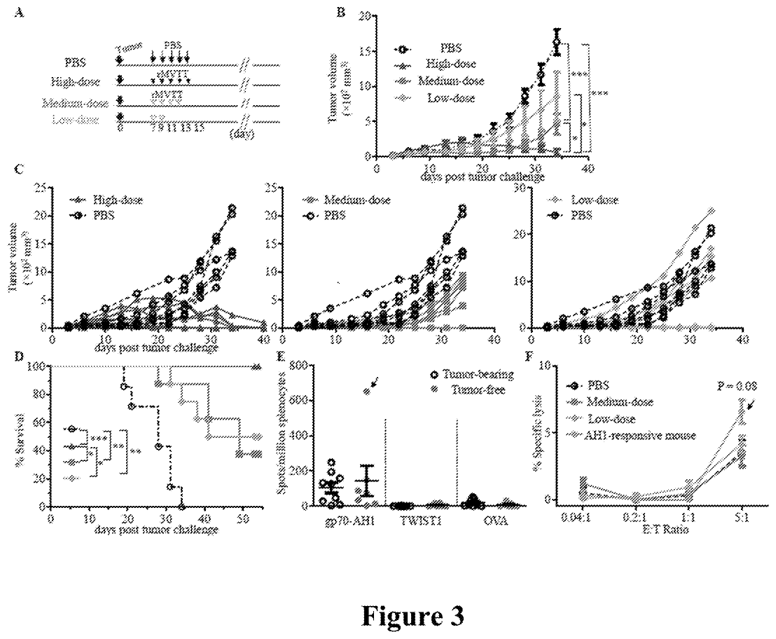

To investigate the ability of rMVTT in treatment of established AB1 mesothelioma in Balb/c mice, the i.t. viral injection was explored as a means to determine its direct antitumor efficacy. Mice were inoculated with AB1 mesothelioma cells 7 days before they received different doses of rMVTT treatment, classified as high-, medium-, low-dose groups (FIG. 3A). The growth of AB1 mesothelioma was significantly inhibited in all mice receiving the rMVTT treatment (FIG. 3B). Furthermore, observations of tumor growth in individual mice showed that high-dose viral treatment completely eliminated tumor growth (FIG. 3C), leading to 100% survival (FIG. 3D), while medium- and low-dose groups showed decreased antitumor efficacy, with only 37.5% mice and 50% stayed tumor-free, respectively (FIGS. 3B-3D), suggesting that rMVTT treatment eliminated established AB1 mesothelioma in a dose-dependent way. The oncolytic effect of rMVTT can create an immune-stimulatory environment to induce immune responses against AB1 tumor antigens. Therefore, two tumor antigens, immunodominant AH1 (gp70₄₂₃₋₄₃₁) and Twist-related protein 1 (TWIST1) peptides, were tested by immunological assays. The peptide gp70-AH1 is a well-characterized immunodominant CTL epitope derived from glycoprotein 70 (gp70) of endogenous murine leukemia virus. The expression of the transcription factor TWIST1 is crucial to tumor's metastatic process and their resistance to drug treatment. Since both gp70-AH1 and TWIST1 were detected in AB1 cells, the existence of antitumor T cells responses was probed by ELlspot and compared between tumor-bearing and tumor-free mice. Splenocytes from only one treated and tumor-free mouse displayed AHI-specific ELlspot response (FIG. 3E) and cytotoxic effect against AB1 cells (FIG. 3F). There was no statistical significance for induction of antitumor T cell responses between tumor-bearing and tumor-free mice (FIGS. 3E-3F). Thus, although rMVTT treatment dose-dependently eliminated established AB1 mesothelioma, the oncolysis of tumors did not readily induce antitumor T cell immunity.

### Example 3—rMVTT Treatment Caused the Accumulation of PMN-MDSCs in TME

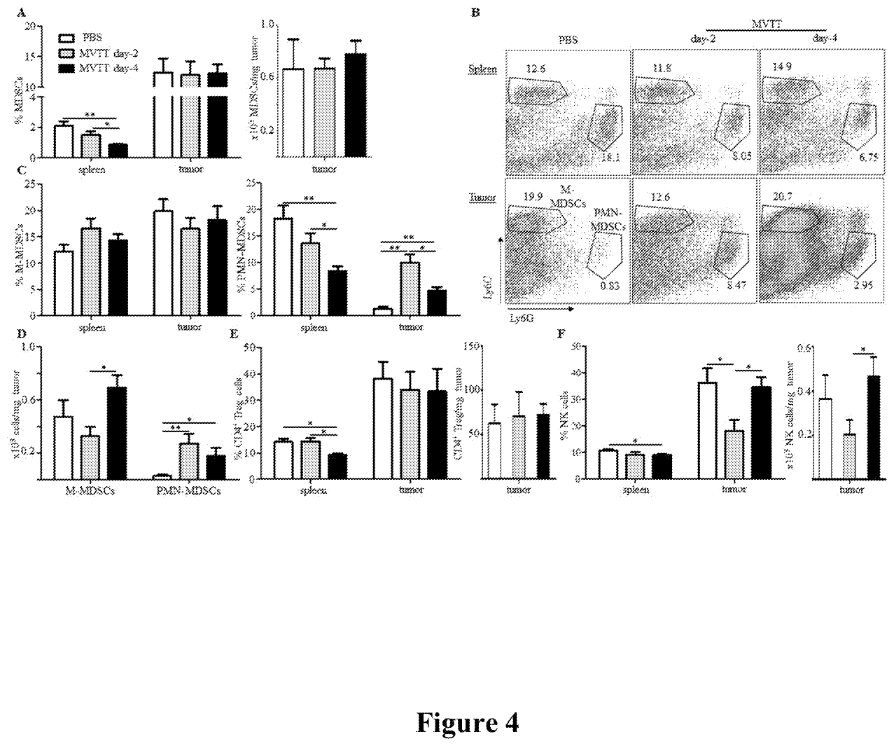

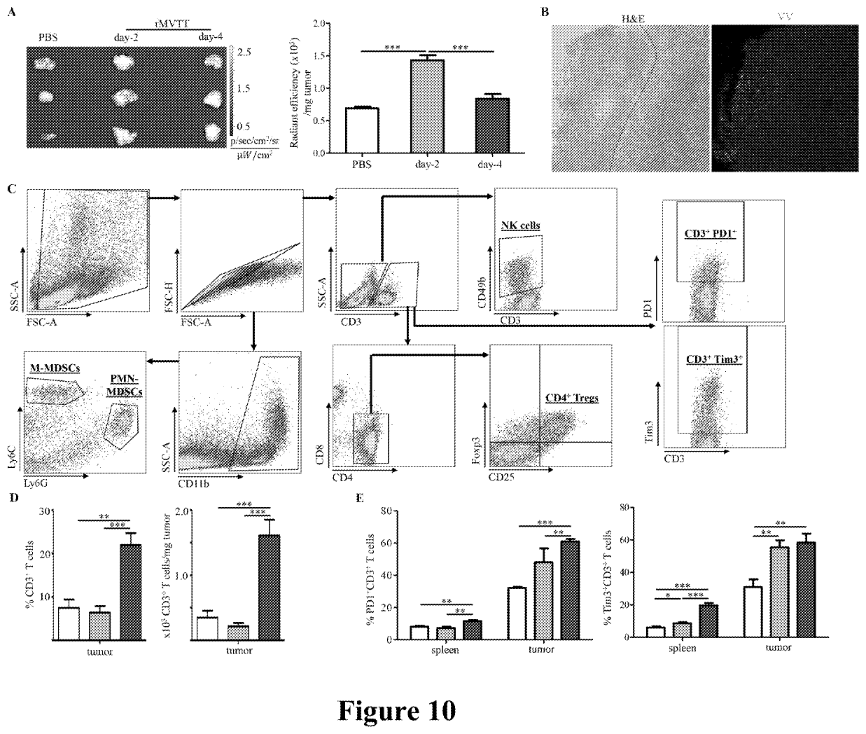

Because the initiation of adaptive antitumor immunity after oncolysis primarily occurs inside the tumor, the TME after rMVTT treatment was examined. At two time-points, 2 and 4 days, after intra-tumoral rMVTT treatment, different tumor resident immune cells, including proportions of CD3⁺ T cells, natural killer (NK) cells, CD4⁺ Treg (CD4⁺ CD25⁺Foxp3⁺) and MDSC subsets (PMN-MDSC, CD11b⁺Ly6G⁺Ly6Clow/int; M-MDSC, CD11b⁺Ly6G-Ly6Chi), and expression of the exhaustion surface markers PD-1 and Tim-3 on CD3⁺ T cells were measured. MDSCs and Tregs are major components of the tumor suppressive microenvironment. The overall levels of MDSCs found in the spleen appeared to decrease over time after rMVTT treatment, while frequencies of tumor-infiltrating MDSCs were maintained at similar levels (FIG. 4A). Two major subsets of MDSCs, PMN-MDSCs and M-MDSCs were then examined because these two subsets displayed remarkable differences in their morphology and suppressive features. Although PMN-MDSCs were largely expanded in peripheral lymphoid organs, M-MDSCs preferentially accumulated inside tumor (FIG. 4B). Furthermore, rMVTT treatment did not influence the frequencies of M-MDSCs either in spleen or in tumor, yet PMN-MDSCs decreased significantly in spleen while increased significantly in tumor over the course of rMVTT treatment (FIGS. 4B-4C). Consistently, in response to rMVTT treatment the absolute PMN-MDSCs cell number also increased significantly in tumor (FIG. 4D). For comparison, although rMVTT treatment decreased the frequencies of CD4⁺ Treg cells in spleen, no significant difference was found in their frequency or cell number in tumor (FIG. 4E). Interestingly, in contrast to the remarkable accumulation of PMN-MDSCs in tumor as early as day-2 post rMVTT treatment, the frequency and cell number of NK cells were significantly decreased (FIG. 4F), implying a possible counteraction between these two cell types. Viral infection-induced inflammatory responses could increase lymphocytes infiltration into the tumor. Indeed, strikingly increased infiltration of CD3⁺ T cells inside tumor was observed at day-4 after rMVTT treatment (FIG. 10D). The increased T cell infiltration, however, was coupled with significantly elevated expression of exhaustion markers PD-1 and Tim-3 (FIG. 10E). Thus, the rMVTT treatment changed local and systemic distribution of a panel of immune cells and, in particular, it resulted in significantly accumulation of PMN-MDSCs in TME.

### Example 4—Trafficking of PMN-MDSCs to the Tumor Site after Intra-Tumoral rMVTT Treatment

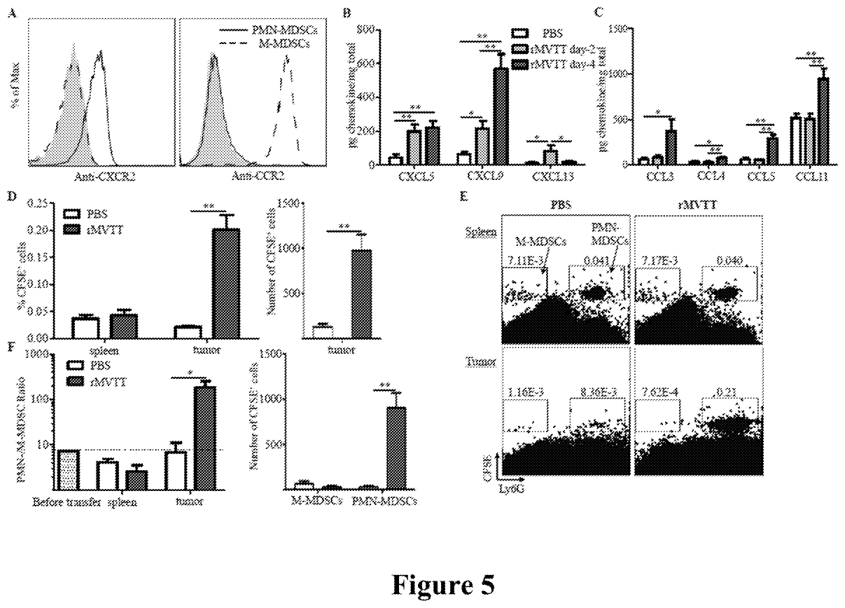

To understand how PMN-MDSCs were recruited into tumors, the role of chemokine induced by rMVTT treatment was examined. Flow cytometric analysis of chemokine receptors revealed that CXCR2 was expressed only on PMN-MDSCs but not on M-MDSCs. Conversely, high level of CCR2 expression was found on M-MDSCs but not on PMN-MDSCs (FIG. 5A). Levels of various chemokines were measured after the rMVTT treatment. A panel of C—X—C chemokines including CXCL5, CXCL9 and CXCL13 were significantly upregulated in tumor as early as 2 days after the treatment (FIG. 5B), whereas upregulated C—C chemokine production was only observed at 4 days (FIG. 5C). These results suggested that CXCR2-expressing PMN-MDSCs might migrate into and adhere to tumor bed primarily in response to the increased C—X—C chemokines. In support of this notion, CFSE-labelled MDSCs derived from mesothelioma-bearing mice were adoptively transferred into recipient mice that were bearing the same tumors but either threated with rMVTT or PBS following the transfer. CFSE labelled MDSCs were quantified in both spleen and tumor by flow cytometry 24 hours after the rMVTT treatment. Compared to PBS-treated recipients, a significant increase in both percentage and absolute number of CFSE⁺ MDSCs in tumors of rMVTT-treated recipients was observed (FIG. 5D). Migrated PMN-MDSCs in tumor were distinguished from M-MDSCs by the expression of Ly6G (FIG. 5E). Moreover, among rMVTT-treated recipients, spleens showed slightly decreased PMN-/M-MDSCs ratios, while their tumors displayed strikingly elevated PMN-/M-MDSCs ratios and absolute numbers of PMN-MDSCs (FIG. 5F). Thus, PMN-MDSCs preferentially migrated from peripheral lymph system into TME in response to chemotaxis induced by the rMVTT treatment.

### Example 5—Disrupting PMN-MDSCs Tumor Trafficking after the rMVTT Treatment

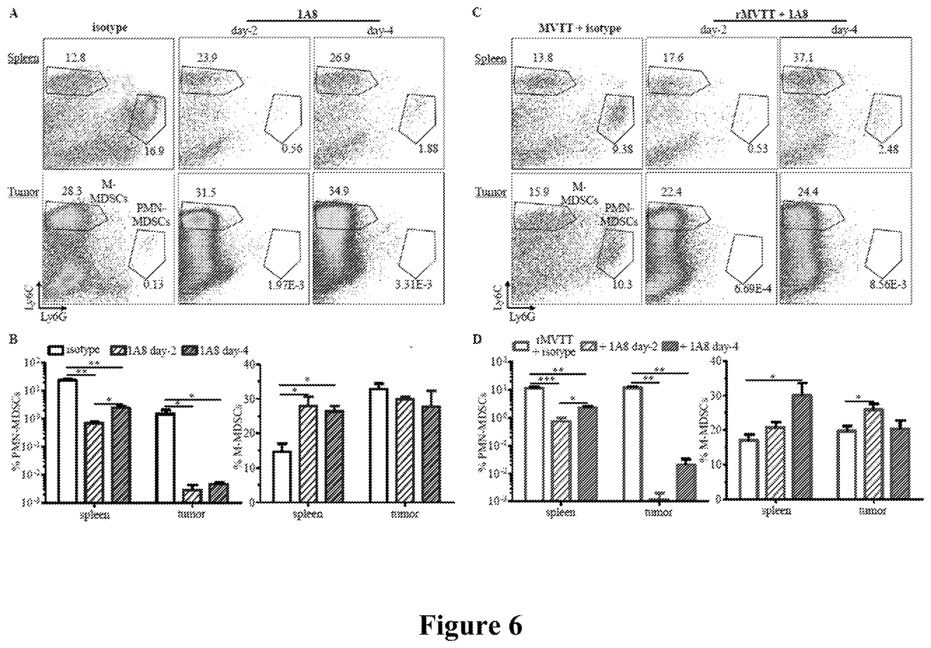

To prevent the migration of MDSCs into tumors, the efficacy of a MDSC depleting antibody, the anti-Ly6G monoclonal antibody 1A8, was tested. Since 1A8 is routinely used to deplete Ly6G⁺ MDSCs, AB1 tumor-bearing mice were treated via the i.t. route with 1A8 or isotype control. Compared with the isotype control, the 1A8-treated mice had significantly decreased frequency of splenic MDSCs yet this antibody did not show efficacy in reducing total MDSCs accumulation in tumors. As expected, however, 1A8 selectively diminished Ly6G⁺ PMN-MDSCs in both spleen and tumor at day-2 after the injection (FIG. 6A). While the effect was maintained in the tumor at day-4, splenic PMN-MDSCs started to reappear (FIGS. 6A-6B). Unlike PMN-MDSCs, the frequency of M-MDSCs in tumor was not affected by 1A8 as a marked increase of splenic M-MDSCs was observed (FIGS. 6A-6B), probably due to continuous generation of M-MDSCs from bone marrow. Subsequently, the impact of 1A8 was investigated in combination with rMVTT. rMVTT treatment resulted in expanded population of PMN-MDSCs in tumors. This expanded population, however, was nearly cleared by 1A8 antibody at day-2 (FIG. 6C). 1A8 also continued to prevent tumor trafficking of PMN-MDSCs at day-4, despite significantly elevated frequency of splenic PMN-MDSCs (FIG. 6D). Thus, the administration of anti-Ly6G 1A8 could specifically disrupt MVTT-induced tumor trafficking of PMN-MDSCs.

### Example 6—Combination of MVTT-Based Oncolysis and PMN-MDSC Depletion Restored Antitumor T Cell Immunity

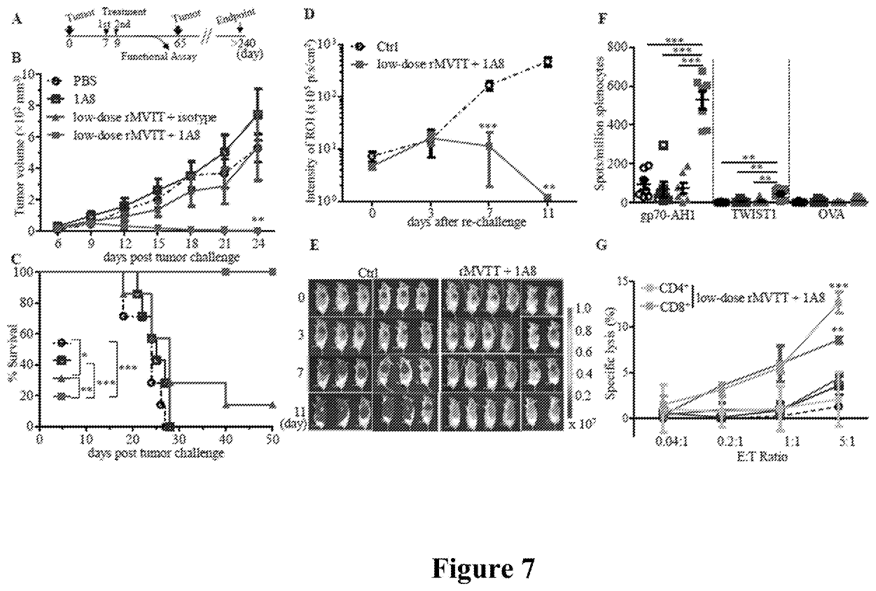

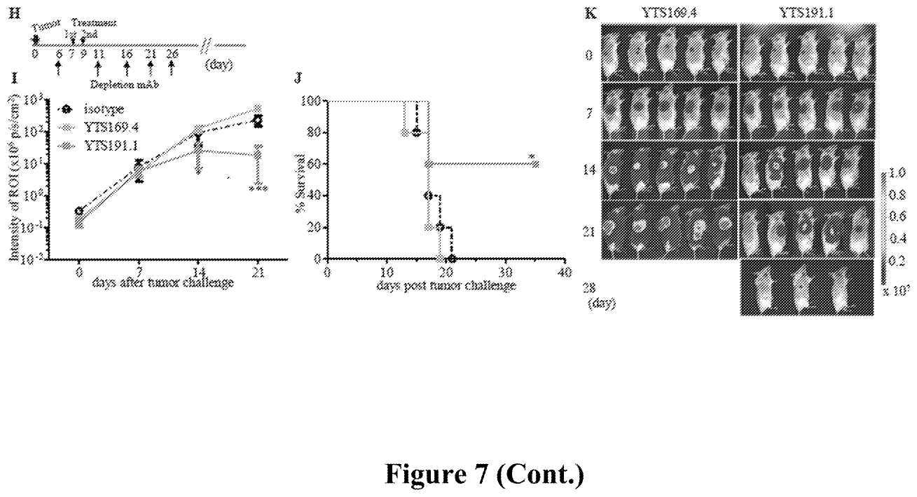

Considering that MDSCs are one of the major types of immunosuppressive cells that inhibit antitumor T cell responses, whether the prevention of MVTT-induced tumor trafficking of PMN-MDSCs would enhance the therapeutic efficacy of the oncolytic viral treatment was examined. In a similar setting as described above, Balb/c mice bearing 7-day-old AB1 tumors were simultaneously injected with rMVTT plus either 1A8 or isotype control. To improve antitumor effect, an additional combination treatment was given 2 days later (FIG. 7A). One time combination treatment slowed tumor growth and resulted tumor regression in 1/7 mice, whereas depleting PMN-MDSCs by 1A8 alone did not impact tumor growth (FIGS. 7B-7C). Critically, the second combination treatment effectively controlled tumor growth and eventually lead to complete elimination of established AB1 mesothelioma (FIGS. 7B-7C). To determine whether prolonged anti-tumor T cell immunity was generated in these controller mice, they were re-challenged with a much higher dose (2×10⁶ cells) of AB1 cells with stable expression of firefly luciferase (AB1-Luc) on their opposite flank 40 days after the complete tumor rejection (FIG. 7A). Complete rejection of AB1-Luc tumors was observed 11 days later in these controller mice, leading to tumor-free survival >30 weeks, while all control mice developed tumors (FIGS. 7D-7E).

Thus, PMN-MDSCs depletion could largely improve the effects of the rMVTT treatment probably by inducing prolonged antitumor immunity. To test this, tumor-specific T cell responses were measured. Murine splenocytes were harvested and tested against tumor antigen either gp70-AH1 or TWIST1 peptides. Significantly increased T cell responses against both gp70-AH1 and TWIST1 were elicited among mice treated with the rMVTT+1A8 combination (FIG. 7F). In vitro CTL assays also demonstrated enhanced CD8⁺ cytotoxic T cells in these mice in comparison to the control groups (FIG. 7G). Furthermore, CD4⁺ or CD8⁺ T cells were depleted using monoclonal antibodies before AB1 tumor-bearing mice received the rMVTT+1A8 combination therapy (FIG. 7H). Remarkably, the depletion of CD8⁺ T cells (YTS169.4) completely diminished the anti-tumor activity of the combination treatment, resulting in rapid tumor outgrowth and all mice died within 21 days. In contrast, the depletion of CD4⁺ T cells (YTS191.1) still preserved therapeutic effects and caused tumor regression in 3/5 mice (FIGS. 7I-7K). Thus, CD8⁺ T cells induced by the rMVTT+1A8 combination are essential for this MVTT-based immuno-oncolytic method. Moreover, the depletion of PMN-MDSCs during localized rMVTT treatment can restore potent systemic antitumor T cell immunity.

### Example 7—PMN-MDSCs Prevent the Induction of Anti-Tumor T Cell Immunity by Restricting Dendritic Cell Activation

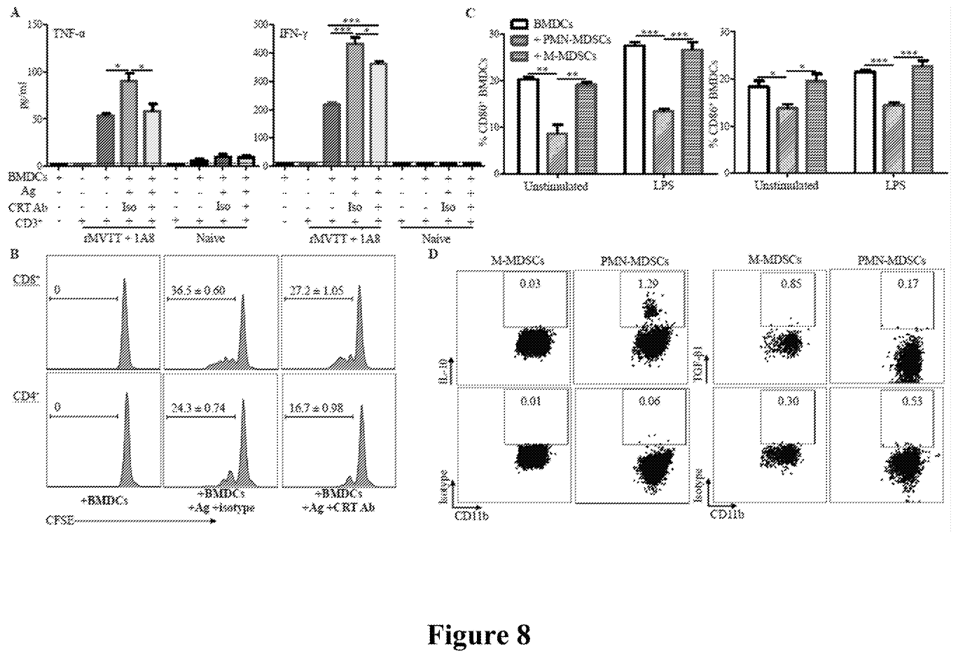

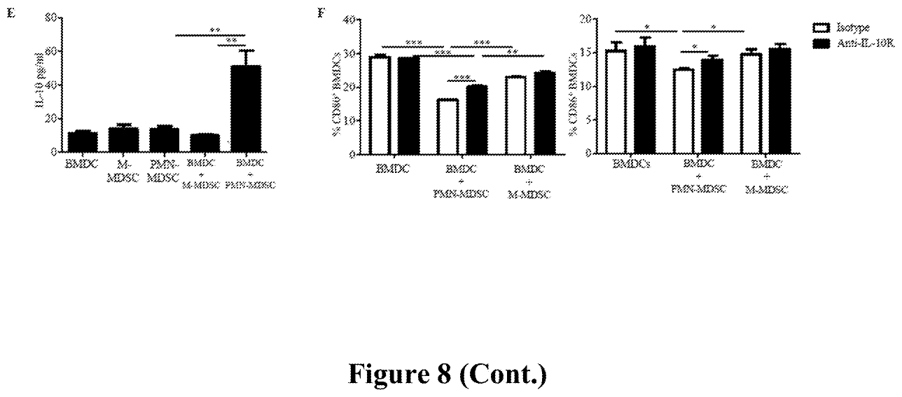

As noted above, MVTT-induced oncolysis of tumors created an immune activating environment with the production of CRT, HMGB1, and ATP. Yet dendritic cells (DCs) failed to recognize and integrate these signals to drive T cell activation. The presence of PMN-MDSCs may supress DC function during MVTT-induced oncolysis of tumors. To test this, the direct impact of PMN-MDSCs on DCs was determined. The ability of bone-marrow derived DCs (BMDCs) in processing and presenting antigens for activating CD3⁺ T cells derived from controller mice that received the MVTT+1A8 combination treatment was determined. MVTT-infected AB1 cell supernatant as a source of tumor antigen pool was used to pulse BMDCs. Antigen-loaded BMDCs greatly enhanced the production of TNF-α and IFN-γ (FIG. 8A) in co-cultures with CD3⁺ T cells of controller mice but not of naïve mice, suggesting T cell activation in response to tumor antigens. Meanwhile, whether surface-exposed CRT proteins would chaperone a wide array of tumor antigens to facilitate their uptake by DCs was also tested. Indeed, the inhibition of this process by an anti-CRT antibody significantly reduced the production of both TNF-α and IFN-γ (FIG. 8A). For confirmation, T cell proliferation was measured. Antigen-pulsed BMDCs could effectively induce both CD4⁺ and CD8⁺ T cell proliferation (FIG. 8B), hence demonstrating activation of tumor antigen-specific T cells. Once again, the presence of anti-CRT antibody could inhibit T cell proliferation (FIG. 8B), suggesting a role of CRT in the activation of DCs-T cell axis. Therefore, in the absence of immunosuppressive environment, oncolysis of tumor cells by the rMVTT were efficient in inducing activation and antigen-presentation of BMDCs.

Subsequently, direct interaction between AB1-induced MDSCs and BMDCs was measured with either culture medium alone or LPS as a maturation signal. As expected, LPS itself significantly increased the level of CD80 expression on BMDCs (P<0.0001, Med vs. LPS) (FIG. 8C). Notably, when MDSCs were present in the co-culture, only the PMN-MDSCs significantly suppressed the expression of CD80 on both unstimulated and LPS-stimulated BMDCs, but not M-MDSCs (FIG. 8C). Whether similar suppressive effect from PMN-MDSCs could be observed was tested in a more relevant model where BMDCs were pulsed with MVTT-infected AB1 cell supernatants other than LPS. Cytokine secretion in the co-culture was measured as a probe for BMDCs activation. BMDCs were more sensitive to PMN-MDSCs-mediated suppression with reduced IL-6 and TNF-α production, compared with M-MDSCs and BMDCs co-cultures. The immunosuppressive cytokine IL-10 is well-known for their ability to block DC maturation process and limit DCs to initiate Th1 response. Indeed, only the PMN-MDSCs exhibited IL-10-producing subsets (FIG. 8D) and released relatively higher IL-10 in the culture. Thus, PMN-MDSCs could directly inhibit DCs activation induced by oncolysis of tumor. Therefore, removal of PMN-MDSCs could rescue DCs functionality for priming adoptive antitumor immunity.

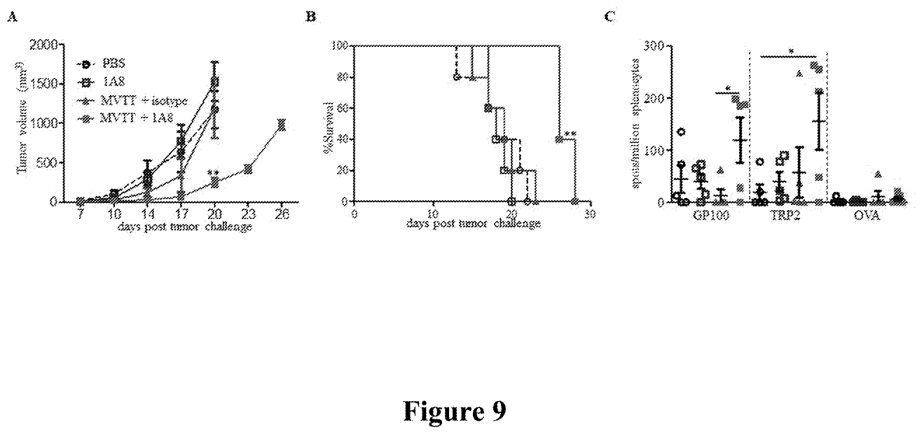

In addition, the effectiveness of the combination therapy was also confirmed in a distinct syngeneic C57BL/6 melanoma model, where enhanced B16F10 tumor regression, prolonged survival and augmented antitumor T cell responses (FIG. 9A-C) were observed, further demonstrating the potency of the MVTT-based immune-oncolytic method.

### Example 8—MVTT Treatment Recruited PMN-MDSCs into TME

Because the initiation of adaptive antitumor immunity after oncolysis primarily occurs inside the tumor, the TME was examined after rMVTT treatment. Analysis of rMVTT-injected AB1 mesothelioma revealed that expression of virus-encoded HcRed was readily detected 2 days after intra-tumoral injection and rapidly decreased thereafter (FIG. 10A). Consistently, immunohistochemical staining of vaccinia viral proteins was only found in tumor tissues at 2 days but not at 4 days after rMVTT treatment, with visible necrotic areas within and adjacent to the zones of infection (FIG. 10B). These results demonstrated rapid but limited rMVTT replication in the TME. Different tumor resident immune cells were then measured, including the proportions of CD3⁺ T cells, natural killer (NK) cells, CD4⁺ Tregs (CD4⁺ CD25⁺Foxp3⁺) and MDSC subsets (PMN-MDSCs, CD11b⁺Ly6G⁺Ly6C^{low/int}; M-MDSCs, CD11b⁺Ly6G⁻Ly6C^{hi}) as well as the expression of the exhaustion surface markers PD-1 and Tim-3 on CD3⁺ T cells by flow cytometry (FIG. 10C). Overall levels of MDSCs in the spleens appeared to decrease over the course of rMVTT treatment, while the frequencies of tumor-infiltrating MDSCs were maintained at similar levels (FIG. 4A). The two major subsets of MDSCs, PMN-MDSCs and M-MDSCs, were examined because they have remarkable differences in their morphology and suppressive activities. PMN-MDSCs were largely expanded in peripheral lymphoid organs, whereas M-MDSCs preferentially accumulated inside tumors of untreated control mice (FIG. 4B). Furthermore, rMVTT treatment did not influence the frequencies of M-MDSCs either in spleens or in tumors; however, PMN-MDSCs decreased significantly in spleens and increased significantly in the TME (FIGS. 4B and 4C). The absolute cell number of PMN-MDSCs in tumors also increased significantly after rMVTT treatment (FIG. 4D). For comparison, although rMVTT treatment decreased the frequencies of CD4⁺ Tregs in the spleen, no significant difference was found in their frequency or cell number in tumors (FIG. 4E). In contrast to the remarkable accumulation of PMN-MDSCs in tumors as early as day 2 post rMVTT treatment, the frequency and cell number of NK cells were significantly decreased (FIG. 4F), implying a possible counteraction between these two cell types. Infection-induced inflammatory responses have been shown to increase lymphocyte infiltration into the TME. Indeed, strikingly increased CD3⁺ T cells were observed inside tumors at day 4 after rMVTT treatment (FIG. 10D). The increased T cell infiltration, however, was coupled with significantly elevated expression of the exhaustion markers PD-1 and Tim-3 (FIG. 10E). Collectively, rMVTT treatment changed the local and systemic distributions of immune cells, particularly the accumulation of PMN-MDSCs in the TME.

### Example 9—Trafficking of PMN-MDSCs into the TME by MVTT-Induced Chemotaxis

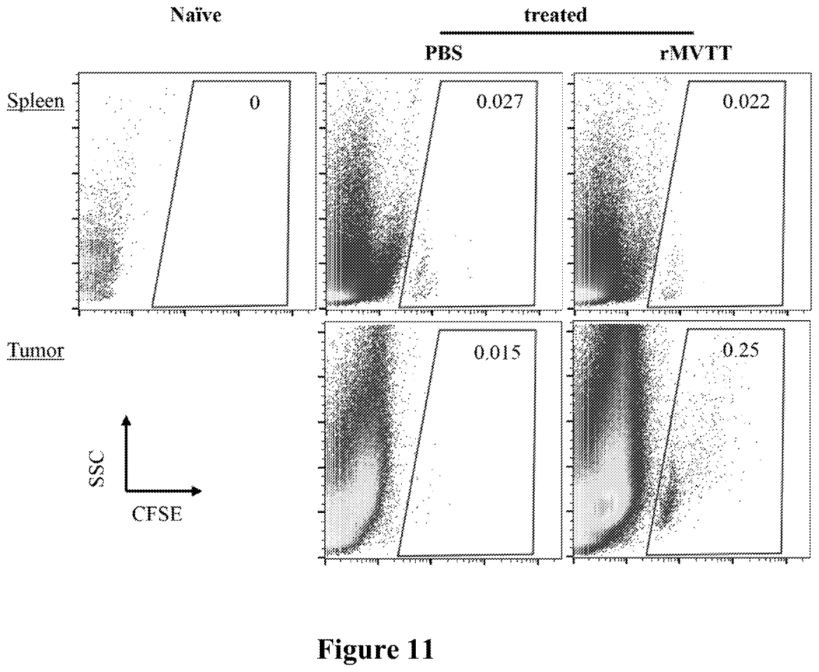

To examine whether PMN-MDSCs may preferentially be recruited to the TME after rMVTT treatment, the expression of chemokine receptors on both MDSC subsets and the levels of chemokines in rMVTT-treated tumors were examined. Flow cytometric analysis of chemokine receptor expression revealed that CXCR2 was expressed only on PMN-MDSCs but not on M-MDSCs. Conversely, high levels of CCR2 expression were found on M-MDSCs but not on PMN-MDSCs (FIG. 5A). The levels of various chemokines were then measured in tumor homogenates after rMVTT treatment. A panel of C—X—C chemokines, including CXCL5, CXCL9 and CXCL13, were significantly upregulated in AB1 mesothelioma as early as 2 days after treatment (FIG. 5B), whereas upregulated C—C chemokine production was only observed 4 days after treatment (FIG. 5C). Thus, CXCR2-expressing PMN-MDSCs might migrate into and adhere to the tumor bed primarily in response to the rapidly increased C—X—C chemokines in the TME. To test this hypothesis, CFSE-labelled MDSCs derived from mesothelioma-bearing mice were adoptively transferred into recipient mice that also bore mesothelioma tumors but were treated with either rMVTT or PBS following the MDSC transfer. CFSE-labelled MDSCs in both the spleen and mesothelioma were then quantified by flow cytometry 24 hours after rMVTT treatment (FIG. 11). Compared to PBS-treated recipients, a significant increase in both the percentage and absolute number of CFSE⁺ MDSCs was observed in tumors of rMVTT-treated recipients (FIG. 5D). Migrated PMN-MDSCs in tumors were distinguished from M-MDSCs by the expression of Ly6G (FIG. 5E). Among the rMVTT-treated recipients, spleens showed slightly decreased PMN-/M-MDSCs ratios, while their tumors displayed strikingly elevated PMN-/M-MDSCs ratios and absolute numbers of PMN-MDSCs (FIGS. 5E and 5F). Overall, PMN-MDSCs preferentially migrated from the peripheral lymph system into the TME in response to chemotaxis induced by rMVTT treatment.

### Example 10—Preferential Depletion of MDSC Subsets by Antibody and Peptibody

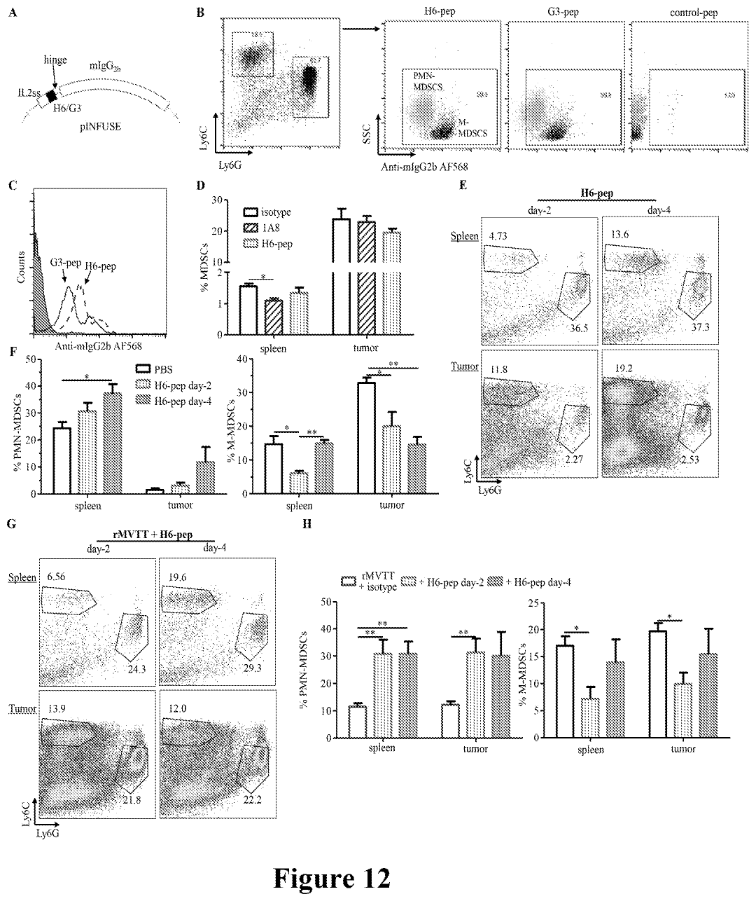

To investigate the role of MDSCs in the rMVTT treatment, two MDSC-depleting agents, anti-Ly6G monoclonal antibody 1A8 and the specific depleting peptibody H6-pep, were explored in our mesothelioma model. 1A8 is routinely used to deplete Ly6G⁺ cells, primarily PMN-MDSCs, whereas H6-pep and G3-pep are two peptibodies with binding specificity to both PMN-MDSCs and M-MDSCs. Accordingly, these two peptibodies were manufactured by a transient expression system in 293F cells using expression plasmids (FIG. 12A). H6-pep showed a relatively higher binding affinity than G3-pep to total MDSCs derived from AB1-mesothelioma-bearing mice (FIGS. 12B and 12C). Therefore, H6-pep was used in the depletion experiments. When AB1 tumor-bearing mice were treated with 1A8 or H6-pep by intra-tumoral injection, only 1A8-treated mice had a significantly decreased frequency of splenic MDSCs, yet both 1A8 and H6-pep did not seem to reduce total MDSC accumulation in tumors (FIG. 12D). However, 1A8 diminished Ly6G⁺ PMN-MDSCs selectively in both spleens and tumors at day 2 after injection (FIGS. 6A and 6B). While this effect was maintained in the tumor at day 4, splenic but not TME PMN-MDSCs started to reappear. Unlike PMN-MDSCs, tumor M-MDSCs were not affected by 1A8, whereas a marked increase in splenic M-MDSCs was observed compared with an isotype control, probably due to the continuous generation of MDSCs from bone marrow. Conversely, with its higher binding affinity to M-MDSCs, H6-pep treatment significantly depleted M-MDSCs but not PMN-MDSCs, especially in the TME; this effect was maintained through day 4 (FIGS. 12E and 12F). Following depletion of M-MDSCs, a significant compensatory increase in the frequency of splenic PMN-MDSCs was observed.

The efficacy of 1A8 and H6-pep during rMVTT treatment was then studied. rMVTT treatment resulted in the increased recruitment of PMN-MDSCs in tumors (FIGS. 6A and 6C). This increased population, however, was nearly cleared by 1A8 antibody treatment at day 2 (FIGS. 6C and 6D). 1A8 also prevented tumor recruitment of PMN-MDSCs at day 4, despite a significantly elevated frequency of splenic PMN-MDSCs. By contrast, H6-pep treatment decreased M-MDSCs while increasing PMN-MDSCs in both the spleens and tumors (FIGS. 12G and 12H). Thus, administration of 1A8 and H6-pep preferentially depleted PMN-MDSCs and M-MDSCs, respectively, and their depletion effects were maintained even after rMVTT administration, which allowed us to study the impact of PMN-MDSCs and M-MDSCs on the induction of antitumor immunity during MVTT-based oncolytic virotherapy.

### Example 11—Depletion of PMN-MDSCs Enhances MVTT Treatment Efficacy by Inducing Antitumor T Cell Immunity

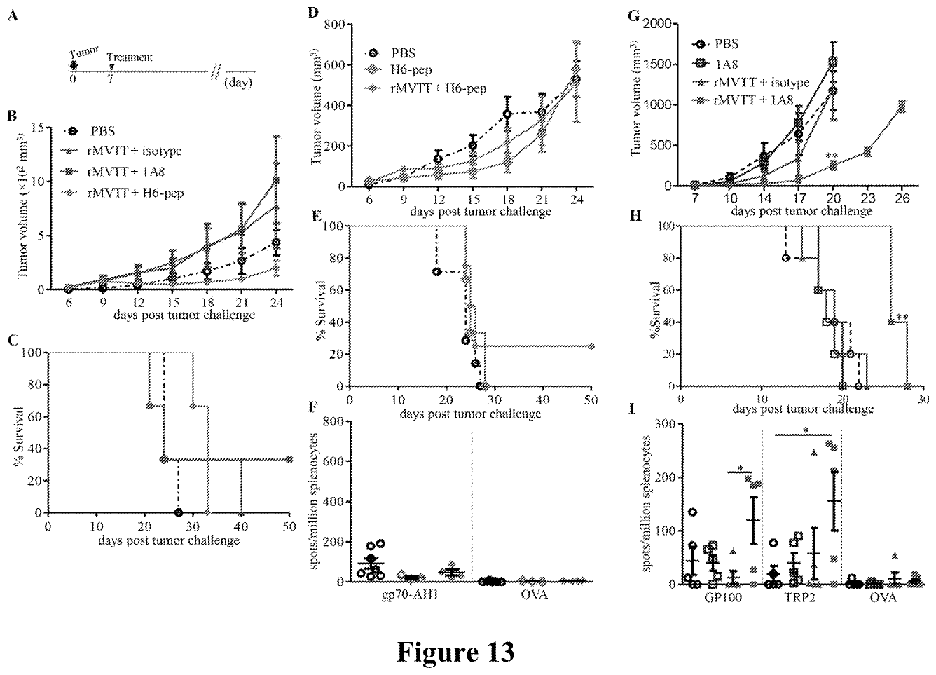

Considering that MDSCs are one of the major immunosuppressive cells that inhibit antitumor T cell responses, whether the depletion of PMN-MDSCs enhanced the therapeutic efficacy of MVTT-based oncolytic virotherapy was explored. In a similar setting as described above, BALB/c mice bearing 7-day-old wild-type AB1 mesothelioma were simultaneously injected with low-dose rMVTT (1×10⁷ PFU) in combination with either 100 μg of 1A8 or H6-pep for the specific depletion of PMN-MDSCs and M-MDSCs, respectively (FIG. 13A). A single delivery of low-dose rMVTT did not control tumor growth. The incorporation of MDSC depletion in this setting, however, did not slow tumor progression or prolong survival (FIGS. 13B and 13C). Given the known dose-dependent effect of the rMVTT treatment, the antitumor effect was supplemented via an additional low-dose 2 days later (FIG. 7A). Two rMVTT treatments alone slowed tumor growth and resulted in tumor regression in 1/7 mice, whereas 1A8 alone did not impact tumor growth at all (FIGS. 7B and 7C). Strikingly, however, the second combined low-dose rMVTT and 1A8 treatment effectively controlled tumor growth and eventually led to complete elimination of established AB1 mesothelioma (FIGS. 7B and 7C). By contrast, the combined rMVTT and H6-pep treatment did not show significant antitumor activity or synergistic effects in mesothelioma elimination (FIGS. 13D and 13E). To determine whether prolonged antitumor T cell immunity was generated in these controller mice, these mice were challenged with a much higher dose (2×10⁶ cells) of AB1-Luc cells on their opposite flank 40 days after complete tumor rejection (FIG. 7A). Complete rejection of AB1-Luc mesothelioma was observed 11 days later in these controller mice, leading to tumor-free survival >30 weeks, while all mice from the control group developed tumors (FIGS. 7D and 7E). These results demonstrated that depletion of PMN-MDSCs but not of M-MDSCs could improve rMVTT treatment efficacy significantly, probably by inducing prolonged antitumor immunity.

To further test this hypothesis, tumor-reactive T cell responses were measured. Murine splenocytes were harvested and tested against gp70-AH1 or TWIST1 peptides (FIG. 7A). The T cell responses against both gp70-AH1 and TWIST1 were significantly increased among mice treated twice with the low-dose rMVTT and 1A8 combination (FIG. 7F). This enhancement was not found with the double rMVTT and H6-pep combination that depleted M-MDSCs (FIG. 13F). In addition, in vitro cytotoxic assays demonstrated enhanced CD8⁺ CTLs in controller mice in comparison to other groups (FIG. 7G). Furthermore, CD4⁺ or CD8⁺ T cells were depleted using the monoclonal antibodies YTS191.1 and YTS169.4, respectively, before AB1 tumor-bearing mice received the rMVTT and 1A8 combination therapy (FIG. 7H). Remarkably, the depletion of CD8⁺ T cells by YTS169.4 completely diminished the antitumor activity of the combination therapy, resulting in uncontrolled tumor outgrowth, and all mice died within 21 days. By contrast, depletion of CD4⁺ T cells by YTS191.1 preserved partial therapeutic effects and caused tumor regression in 3/5 mice (FIGS. 7I-7K). To determine whether this discovery could be applied to other malignant tumors, the efficacy of the combined rMVTT and 1A8 therapy was tested in a distinct syngeneic C57BL/6 melanoma model. Similarly, this combination therapy resulted in enhanced B16F10 tumor regression, prolonged survival and augmented antitumor T cell responses (FIGS. 13G-13I). Collectively, depletion of PMN-MDSCs during localized MVTT-based oncolytic virotherapy elicited potent systemic and long lasting antitumor T cell immunity.

### Example 12—PMN-MDSCs Prevent the Induction of Antitumor T Cell Immunity by Restricting Dendritic Cell Activation

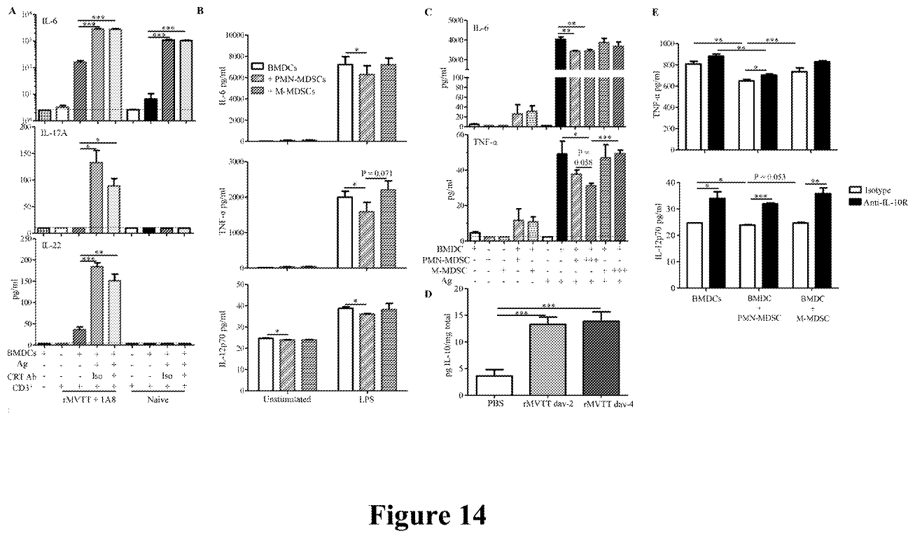

Although rMVTT-induced oncolysis created an immune-activating environment with the production of CRT, HMGB1 and ATP, anti-mesothelioma specific T cell responses were not readily induced (FIGS. 3E and 3F). This situation, however, was completely changed when PMN-MDSCs were depleted during the rMVTT treatment (FIGS. 7F and 7G). Therefore, PMN-MDSCs might have suppressive effects on DCs through direct cross-talk in the TME of our model. To test this possibility, the direct impact of PMN-MDSCs on DCs was examined. First the ability was tested of bone marrow-derived DCs (BMDCs) to process and present antigens for activating CD3⁺ T cells derived from controller mice that received combined rMVTT and 1A8 treatment. rMVTT-treated AB1 cell supernatants were used as a supply of tumor antigens to pulse BMDCs. Remarkably increased was observed in the production of the proinflammatory cytokine IL-6 in co-cultures when BMDCs were pulsed with antigens (FIG. 14A). Meanwhile, antigen-loaded BMDCs greatly enhanced the production of TNF-α and IFN-γ (FIG. 8A), as well as the Th17 cytokines IL-17A and IL-22 (FIG. 14A), in co-cultures with CD3⁺ T cells of controller mice but not of naïve mice, suggesting T cell activation in response to tumor antigens. Previously, surface-exposed CRT protein has been shown to chaperone tumor antigens to facilitate their uptake by DCs. Indeed, an anti-CRT antibody significantly reduced the production of both TNF-α and IFN-γ (FIG. 8A). To confirm these findings, T cell proliferation was measured. Antigen-pulsed BMDCs effectively induced controller CD4⁺ and CD8⁺ T cell proliferation (FIG. 8B), demonstrating activation of tumor antigen-specific T cells. Once again, the presence of an anti-CRT antibody inhibited T cell proliferation (FIG. 8B), suggesting a role for CRT in the activation of the DC-T cell axis. Therefore, in the absence of PMN-MDSCs, rMVTT-induced CRT exposure enhances the activation of BMDCs to elicit potent antitumor T cell immunity.

Subsequently, the direct interaction between AB1-induced MDSCs and BMDCs was measured. BMDCs were co-cultured with AB1-induced MDSCs in the presence or absence of LPS. CD80 and CD86 expression on BMDCs was significantly upregulated by LPS stimulation (P<0.001 for CD80, P<0.05 for CD86, Unstimulated versus LPS), suggesting BMDC maturation (FIG. 8C). Notably, when MDSCs were present in the co-culture, PMN-MDSCs but not M-MDSCs significantly suppressed expression of CD80 and CD86 on both unstimulated and LPS-stimulated BMDCs (FIG. 8C). LPS-induced changes in cytokine production were also analyzed. Supernatants collected from BMDCs without LPS showed very low levels of cytokines consistently. In contrast, culture supernatants with LPS resulted in marked increases of the proinflammatory cytokines IL-6 and TNF-α, as well as type 1 cytokine IL-12p70 (FIG. 14B). In consistency with PMN-MDSC's ability of down-regulating BMDC activation, the presence of PMN-MDSCs in the co-culture significantly inhibited the induction of IL-6, TNF-α and IL-12p70, further supporting the role of PMN-MDSCs in suppressing BMDCs activation (FIG. 14B). Whether PMN-MDSCs have similar suppressive effects when BMDCs were pulsed with rMVTT-treated AB1 cell supernatants rather than LPS was then tested. By measuring cytokines related to BMDC activation, PMN-MDSCs but not M-MDSCs significantly inhibited IL-6 and TNF-α production in co-cultures, and the inhibitory effect of PMN-MDSCs on TNF-α production was dose-dependent (FIG. 14C).

To understand the underlying mechanism of PMN-MDSC-mediated immunosuppression, productions of IL-10 and TGF-β in MDSC subsets were examined. MDSCs did not produce TGF-β and only PMN-MDSCs exhibited an IL-10-producing subset (FIG. 8D). Furthermore, the production of IL-10 was enhanced when PMN-MDSCs were co-cultured with BMDCs in vitro (FIG. 8E) as well as following intra-tumoral MVTT treatment in vivo (FIG. 14D). The immunosuppressive cytokine IL-10 is well-known to inhibit DC maturation and prevent DCs from initiating Th1 responses. Crosstalk between MDSC and macrophage has been reported to reduce macrophage production of IL-12 and increase MDSC production of IL-10 to promote tumor progression. Therefore, the suppressive capacity of PMN-MDSCs may depend on their IL-10 production. To test this, purified PMN-MDSCs or M-MDSCs derived from AB1-bearing mice were co-cultured with LPS-activated BMDCs in the presence of IL-10 receptor blocking antibody or isotype control. Compared the expression of activation markers on BMDCs, the presence of PMN-MDSCs consistently down-regulated CD80 and CD86 expression on BMDCs (FIG. 8F). However, PMN-MDSC-mediated suppression can be partially alleviated by the blockade of IL-10 receptor (FIG. 8F). In addition, secreted cytokines in the supernatant were examined and blocking IL-10 receptor also significantly elevated production of TNF-α and IL-12p70 (FIG. 14E), suggesting IL-10 production by PMN-MDSCs appeared to be a direct means of suppression in our in vitro suppression assay. Collectively, while rMVTT treatments facilitate CRT-dependent antigen uptake, as well as activation and antigen-presentation of BMDCs, PMN-MDSCs likely directly inhibit DC activation and lead to the reduced efficacy or failure of oncolytic viral treatment.

### REFERENCES

1. Yap T A, Aerts J G, Popat S, Fennell D A. Novel insights into mesothelioma biology and implications for therapy. Nat Rev Cancer 2017; 17:475-88.
2. Rusch V W. Pemetrexed and cisplatin for malignant pleural mesothelioma: a new standard of care? J Clin Oncol 2003; 21:2629-30.
3. Dozier J, Zheng H, Adusumilli P S. Immunotherapy for malignant pleural mesothelioma: current status and future directions. Transl Lung Cancer Res 2017; 6:315-24.
4. Fukuhara H, Ino Y, Todo T. Oncolytic virus therapy: A new era of cancer treatment at dawn. Cancer Sci 2016; 107:1373-9.
5. Kaufman H L, Kohlhapp F J, Zloza A. Oncolytic viruses: a new class of immunotherapy drugs. Nat Rev Drug Discov 2015; 14:642-62.
6. Zamarin D, Holmgaard R B, Subudhi S K, Park J S, Mansour M, Palese P, et al. Localized Oncolytic Virotherapy Overcomes Systemic Tumor Resistance to Immune Checkpoint Blockade Immunotherapy. Sci Transl Med 2014; 6.
7. Ranki T, Pesonen S, Hemminki A, Partanen K, Kairemo K, Alanko T, et al. Phase I study with ONCOS-102 for the treatment of solid tumors—an evaluation of clinical response and exploratory analyses of immune markers. J Immunother Cancer 2016; 4:17.
8. Hanahan D, Weinberg R A. Hallmarks of cancer: the next generation. Cell 2011; 144:646-74. 9. Scarlett U K, Rutkowski M R, Rauwerdink A M, Fields J, Escovar-Fadul X, Baird J, et al. Ovarian cancer progression is controlled by phenotypic changes in dendritic cells. J Exp Med 2012; 209:495-506.
9. Scarlett U K, Rutkowski M R, Rauwerdink A M, Fields J, Escovar-Fadul X, Baird J, et al. Ovarian cancer progression is controlled by phenotypic changes in dendritic cells. J Exp Med 2012; 209:495-506.
10. Bronte V, Brandau S, Chen S H, Colombo M P, Frey A B, Greten T F, et al. Recommendations for myeloid-derived suppressor cell nomenclature and characterization standards. Nat Commun 2016; 7:12150.
11. Kaufman H L, Kim D W, DeRaffele G, Mitcham J, Coffin R S, Kim-Schulze S. Local and distant immunity induced by intralesional vaccination with an oncolytic herpes virus encoding GM-CSF in patients with stage Mc and IV melanoma. Ann Surg Oncol 2010; 17:718-30.
12. Ranki T, Joensuu T, Jager E, Karbach J, Wahle C, Kairemo K, et al. Local treatment of a pleural mesothelioma tumor with ONCOS-102 induces a systemic antitumor CD8+ T-cell response, prominent infiltration of CD8+ lymphocytes and Th1 type polarization. Oncoimmunology 2014; 3:e958937.
13. Cerullo V, Pesonen S, Diaconu I, Escutenaire S, Arstila P T, Ugolini M, et al. Oncolytic adenovirus coding for granulocyte macrophage colony-stimulating factor induces antitumoral immunity in cancer patients. Cancer Res 2010; 70:4297-309.
14. Ribas A, Dummer R, Puzanov I, VanderWalde A, Andtbacka R H I, Michielin O, et al. Oncolytic Virotherapy Promotes Intratumoral T Cell Infiltration and Improves Anti-PD-1 Immunotherapy. Cell 2017; 170:1109-19 e10.
15. Tan Z, Zhou J, Cheung A K, Yu Z, Cheung K W, Liang J, et al. Vaccine-elicited CD8+ T cells cure mesothelioma by overcoming tumor-induced immunosuppressive environment. Cancer Res 2014; 74:6010-21.
16. Yu Z, Tan Z, Lee B K, Tang J, Wu X, Cheung K W, et al. Antigen spreading-induced CD8+ T cells confer protection against the lethal challenge of wild-type malignant mesothelioma by eliminating myeloid-derived suppressor cells. Oncotarget 2015; 6:32426-38.
17. Hou W, Sampath P, Rojas J J, Thorne S H. Oncolytic Virus-Mediated Targeting of PGE2 in the Tumor Alters the Immune Status and Sensitizes Established and Resistant Tumors to Immunotherapy. Cancer Cell 2016; 30:108-19.
18. Veltman J D, Lambers M E H, van Nimwegen M, Hendriks R W, Hoogsteden H C, Aerts JGJV, et al. COX-2 inhibition improves immunotherapy and is associated with decreased numbers of myeloid-derived suppressor cells in mesothelioma. Celecoxib influences MDSC function. Bmc Cancer 2010; 10.
19. Yamada N, Oizumi S, Kikuchi E, Shinagawa N, Konishi-Sakakibara J, Ishimine A, et al. CD8+ tumor-infiltrating lymphocytes predict favorable prognosis in malignant pleural mesothelioma after resection. Cancer Immunol Immunother 2010; 59:1543-9.
20. Krysko D V, Garg A D, Kaczmarek A, Krysko O, Agostinis P, Vandenabeele P. Immunogenic cell death and DAMPs in cancer therapy. Nat Rev Cancer 2012; 12:860-75.
21. Facciponte J G, Ugel S, De Sanctis F, Li C, Wang L, Nair G, et al. Tumor endothelial marker 1-specific DNA vaccination targets tumor vasculature. J Clin Invest 2014; 124:1497-511.
22. Kreiter S, Vormehr M, van de Roemer N, Diken M, Lower M, Diekmann J, et al. Mutant WIC class II epitopes drive therapeutic immune responses to cancer. Nature 2015; 520:692-6.
23. Qin Q, Xu Y, He T, Qin C, Xu J. Normal and disease-related biological functions of Twistl and underlying molecular mechanisms. Cell Res 2012; 22:90-106.
24. Gabrilovich D I, Ostrand-Rosenberg S, Bronte V. Coordinated regulation of myeloid cells by tumours. Nat Rev Immunol 2012; 12:253-68.
25. Youn J I, Gabrilovich D I. The biology of myeloid-derived suppressor cells: the blessing and the curse of morphological and functional heterogeneity. Eur J Immunol 2010; 40:2969-75.
26. Fortin C, Huang X P, Yang Y P. NK Cell Response to Vaccinia Virus Is Regulated by Myeloid-Derived Suppressor Cells. Journal of Immunology 2012; 189:1843-9.
27. Hoechst B, Voigtlaender T, Ormandy L, Gamrekelashvili J, Zhao F, Wedemeyer H, et al. Myeloid derived suppressor cells inhibit natural killer cells in patients with hepatocellular carcinoma via the NKp30 receptor. Hepatology 2009; 50:799-807.
28. Zamarin D, Holmgaard R B, Ricca J, Plitt T, Palese P, Sharma P, et al. Intratumoral modulation of the inducible co-stimulator ICOS by recombinant oncolytic virus promotes systemic anti-tumour immunity. Nat Commun 2017; 8:14340.
29. Singh R, Lillard J W, Jr., Singh S. Chemokines: key players in cancer progression and metastasis. Front Biosci (Schol Ed) 2011; 3:1569-82.
30. Highfill S L, Cui Y, Giles A J, Smith J P, Zhang H, Morse E, et al. Disruption of CXCR2-mediated MDSC tumor trafficking enhances anti-PD1 efficacy. Sci Transl Med 2014; 6:237ra67.
31. Qin H, Lerman B, Sakamaki I, Wei G W, Cha S C C, Rao S S, et al. Generation of a new therapeutic peptide that depletes myeloid-derived suppressor cells in tumor-bearing mice. Nat Med 2014; 20:676-81.
32. Ostrand-Rosenberg S, Sinha P, Beury D W, Clements V K. Cross-talk between myeloid-derived suppressor cells (MDSC), macrophages, and dendritic cells enhances tumor-induced immune suppression. Semin Cancer Biol 2012; 22:275-81.
33. Helft J, Bottcher J, Chakravarty P, Zelenay S, Huotari J, Schraml B U, et al. GM-CSF Mouse Bone Marrow Cultures Comprise a Heterogeneous Population of CD11c(+)MHCII(+) Macrophages and Dendritic Cells. Immunity 2015; 42:1197-211.
34. Basu S, Srivastava P K. Calreticulin, a peptide-binding chaperone of the endoplasmic reticulum, elicits tumor- and peptide-specific immunity. J Exp Med 1999; 189:797-802.
35. Kumar V, Patel S, Tcyganov E, Gabrilovich D I. The Nature of Myeloid-Derived Suppressor Cells in the Tumor Microenvironment. Trends Immunol 2016; 37:208-20.
36. Schmidt S V, Nino-Castro A C, Schultze J L. Regulatory dendritic cells: there is more than just immune activation. Front Immunol 2012; 3.
37. Sinha P, Clements V K, Bunt S K, Albelda S M, Ostrand-Rosenberg S. Cross-talk between myeloid-derived suppressor cells and macrophages subverts tumor immunity toward a type 2 response. Journal of Immunology 2007; 179:977-83.
38. Beury D W, Parker K H, Nyandjo M, Sinha P, Carter K A, Ostrand-Rosenberg S. Cross-talk among myeloid-derived suppressor cells, macrophages, and tumor cells impacts the inflammatory milieu of solid tumors. J Leukocyte Biol 2014; 96:1109-18.
39. Andtbacka R H, Kaufman H L, Collichio F, Amatruda T, Senzer N, Chesney J, et al. Talimogene Laherparepvec Improves Durable Response Rate in Patients With Advanced Melanoma. J Clin Oncol 2015; 33:2780-8.
40. Kohlhapp F J, Kaufman H L. Molecular Pathways: Mechanism of Action for Talimogene Laherparepvec, a New Oncolytic Virus Immunotherapy. Clin Cancer Res 2016; 22:1048-54.
41. Pol J, Buque A, Aranda F, Bloy N, Cremer I, Eggermont A, et al. Trial Watch-Oncolytic viruses and cancer therapy. Oncoimmunology 2016; 5:e1117740.
42. Krug L M, Zauderer M G, Adusumili P S, Mcgee E, Sepkowitz K, Klang M, et al. Phase I study of intra-pleural administration of GL-ONC1, an oncolytic vaccinia virus, in patients with malignant pleural effusion. Journal of Clinical Oncology 2015; 33.
43. Boisgerault N, Achard C, Delaunay T, Cellerin L, Tangy F, Gregoire M, et al. Oncolytic virotherapy for human malignant mesothelioma: recent advances. Oncolytic Virother 2015; 4:133-40.
44. Diaz R M, Galivo F, Kottke T, Wongthida P, Qiao J, Thompson J, et al. Oncolytic immunovirotherapy for melanoma using vesicular stomatitis virus. Cancer Research 2007; 67:2840-8.
45. Tai L H, de Souza C T, Belanger S, Ly L, Alkayyal A A, Zhang J Q, et al. Preventing Postoperative Metastatic Disease by Inhibiting Surgery-Induced Dysfunction in Natural Killer Cells. Cancer Research 2013; 73:97-107.
46. Ochoa A C, Zea AH, Hernandez C, Rodriguez P C. Arginase, prostaglandins, and myeloid-derived suppressor cells in renal cell carcinoma. Clin Cancer Res 2007; 13:721s-6s.
47. Diaz-Montero C M, Salem M L, Nishimura M I, Garrett-Mayer E, Cole D J, Montero A J. Increased circulating myeloid-derived suppressor cells correlate with clinical cancer stage, metastatic tumor burden, and doxorubicin-cyclophosphamide chemotherapy. Cancer Immunol Immunother 2009; 58:49-59.
48. Almand B, Clark J I, Nikitina E, van Beynen J, English N R, Knight S C, et al. Increased production of immature myeloid cells in cancer patients: a mechanism of immunosuppression in cancer. J Immunol 2001; 166:678-89.
49. Lesokhin A M, Hohl T M, Kitano S, Cortez C, Hirschhorn-Cymerman D, Avogadri F, et al. Monocytic CCR2(+) myeloid-derived suppressor cells promote immune escape by limiting activated CD8 T-cell infiltration into the tumor microenvironment. Cancer Res 2012; 72:876-86.
50. Liu Z, Ravindranathan R, Kalinski P, Guo Z S, Bartlett D L. Rational combination of oncolytic vaccinia virus and PD-L1 blockade works synergistically to enhance therapeutic efficacy. Nat Commun 2017; 8:14754.
51. Srivastava M K, Zhu L, Harris-White M, Kar U K, Huang M, Johnson M F, et al. Myeloid suppressor cell depletion augments antitumor activity in lung cancer. PLoS One 2012; 7:e40677.
52. Stromnes I M, Brockenbrough J S, Izeradjene K, Carlson M A, Cuevas C, Simmons R M, et al. Targeted depletion of an MDSC subset unmasks pancreatic ductal adenocarcinoma to adaptive immunity. Gut 2014; 63:1769-81.
53. Lesterhuis W J, Salmons J, Nowak A K, Rozali E N, Khong A, Dick I M, et al. Synergistic effect of CTLA-4 blockade and cancer chemotherapy in the induction of anti-tumor immunity. PLoS One 2013; 8:e61895.
54. Glodde N, Bald T, van den Boorn-Konijnenberg D, Nakamura K, O'Donnell J S, Szczepanski S, et al. Reactive Neutrophil Responses Dependent on the Receptor Tyrosine Kinase c-MET Limit Cancer Immunotherapy. Immunity 2017; 47:789-802 e9.
55. Van Valckenborgh E, Schouppe E, Movahedi K, De Bruyne E, Menu E, De Baetselier P, et al. Multiple myeloma induces the immunosuppressive capacity of distinct myeloid-derived suppressor cell subpopulations in the bone marrow. Leukemia 2012; 26:2424-8.
56. Noman M Z, Desantis G, Janji B, Hasmim M, Karray S, Dessen P, et al. PD-L1 is a novel direct target of HIF-lalpha, and its blockade under hypoxia enhanced MDSC-mediated T cell activation. J Exp Med 2014; 211:781-90.
57. Huang B, Pan P Y, Li Q, Sato A I, Levy D E, Bromberg J, et al. Gr-1+CD115+ immature myeloid suppressor cells mediate the development of tumor-induced T regulatory cells and T-cell anergy in tumorbearing host. Cancer Res 2006; 66:1123-31.
58. De Santo C, Arscott R, Booth S, Karydis I, Jones M, Asher R, et al. Invariant NKT cells modulate the suppressive activity of IL-10-secreting neutrophils differentiated with serum amyloid A. Nat Immunol 2010; 11:1039-46.
59. Meyers D E, Wang A A, Thirukkumaran C M, Morris D G. Current Immunotherapeutic Strategies to Enhance Oncolytic Virotherapy. Front Oncol 2017; 7:114.
60. Lawson K A, Mostafa A A, Shi Z Q, Spurrell J, Chen W, Kawakami J, et al. Repurposing Sunitinib with Oncolytic Reovirus as a Novel Immunotherapeutic Strategy for Renal Cell Carcinoma. Clin Cancer Res 2016; 22:5839-50.
61. Yamanaka M, Tada Y, Kawamura K, Li Q, Okamoto S, Chai K, et al. E1B-55 kDa-defective adenoviruses activate p53 in mesothelioma and enhance cytotoxicity of anticancer agents. J Thorac Oncol 2012; 7:1850-7.
62. Zhou J, Liu M, Sun H, Feng Y, Xu L, Chan A W H, et al. Hepatoma-intrinsic CCRK inhibition diminishes myeloid-derived suppressor cell immunosuppression and enhances immune-checkpoint blockade efficacy. Gut 2017.
63. Yu W, Fang Q, Zhu W, Wang H, Tien P, Zhang L, et al. One time intranasal vaccination with a modified vaccinia Tiantan strain MVTT(ZCI) protects animals against pathogenic viral challenge. Vaccine 2010; 28:2088-96.

1. A method of treating a cancer in a subject, comprising administering to the subject a combination of an oncolytic virus and a therapy that induces depletion of tumor-induced bone marrow myeloid-derived suppressor cells of polymorphonuclear type (PMN-MDSCs).

2. The method of claim 1, wherein the oncolytic virus is an adenovirus, reovirus, herpes virus, picornavirus, paramyxovirus, parvovirus, rhabdovirus, or vaccinia virus.

3. The method of claim 1, wherein the oncolytic virus is replication competent.

4. The method of claim 1, wherein the oncolytic virus is replication incompetent.

5. The method of claim 1, wherein the oncolytic virus is a replication-attenuated vaccinia virus.

6. The method of claim 5, wherein the replication incompetent vaccinia virus is a modified vaccinia TianTan (MVTT) virus having a deletion of the viral M1L-K2L genes.

7. The method of claim 6, wherein the MVTT comprises a heterologous polynucleotide that replaces the deleted viral M1L-K2L.

8. The method of claim 1, wherein the therapy that induces depletion of tumor-induced PMN-MDSCs specifically induces depletion of tumor-induced PMN-MDSCs or induced M-MDSCs.

9. The method of claim 1, wherein the therapy that induces depletion of tumor-induced PMN-MDSCs comprises administering to the subject gemcitabine, fluorouracil, bindarit, PDE5 inhibitors, tadalafil, nitroaspirin, COX-2 inhibitors, ipilimumab, bevacizumab, celecoxib, a combination of sildenafil and tadalafil, N-hydroxy-L-arginine, N-acetyl cysteine (NAC), CpG oligodeoxy-nucleotides (ODN), Bardoxolone methyl (CDDO-Me), Withaferin A, monoclonal anti-Gr1 antibody, IL4Ra aptamer, peptibodies that target MDSC-membrane proteins, or an antibody against lymphocyte antigen 6 complex locus G6D (Ly6G).

10. The method of claim 1, wherein the therapy that induces depletion of tumor-induced PMN-MDSCs comprises administering an antibody.

11. The method of claim 10, wherein the antibody is against MDSC.

12. The method of claim 1, wherein the oncolytic virus and/or the therapy that induces depletion of tumor-induced PMN-MDSCs is administered multiple times over a period of two to fourteen days.

13. The method of claim 1, wherein the oncolytic virus and/or the therapy that induces depletion of tumor-induced PMN-MDSCs is administered by an intra-tumoral injection.

14. The method of claim 1, further comprising administering one or more additional anti-cancer therapies to the subject.

15. The method of claim 14, wherein the one or more additional anti-cancer therapies comprise administering a chemotherapeutic drug, a check-point inhibitor, adjuvant, anemia drug, radiation therapy, stem cell transplant, chimeric antigen receptor (CAR)-expressing T-cells (CAR T-cells), or a combination of two or more of the foregoing.

16. The method of claim 15, wherein the check-point inhibitor is an inhibitor of: cytotoxic T-lymphocyte antigen 4 (CTLA4), programmed cell death protein 1 (PD-1) and programmed cell death 1 ligand 1 (PD-L1).

17. The method of claim 16, wherein the inhibitor of CTLA4 is an antibody that binds CTLA4, the inhibitor of PD-1 is an antibody that binds PD-1, and the inhibitor of PD-L1 is an antibody that binds to PD-L1.

18. The method of claim 1, wherein the subject is a human.

19. The method of claim 1, wherein the cancer is mesothelioma, melanoma or other solid tumors.

20. A composition comprising an oncolytic virus and a product that induces depletion of tumor-induced bone marrow myeloid-derived suppressor cells of polymorphonuclear type (PMN-MDSCs) and a pharmaceutically acceptable carrier.

21. The composition of claim 20, wherein the oncolytic virus is an adenovirus, reovirus, herpes virus, picornavirus, paramyxovirus, parvovirus, rhabdovirus, or vaccinia virus.

22. The composition of claim 20, wherein the oncolytic virus is replication competent.

23. The composition of claim 20, wherein the oncolytic virus is replication incompetent.

24. The composition of claim 20, wherein the oncolytic virus is a replication-attenuated vaccinia virus.

25. The composition of claim 24, wherein the replication incompetent vaccinia virus is a modified vaccinia TianTan (MVTT) virus having a deletion of the viral M1L-K2L genes.

26. The composition of claim 25, wherein the MVTT comprises a heterologous polynucleotide that replaces the deleted viral M1L-K2L.

27. The composition of claim 20, wherein the compound that induces depletion of tumor-induced PMN-MDSCs specifically induces depletion of tumor-induced PMN-MDSCs without affecting tumor-induced M-MDSCs.

28. The composition of claim 20, wherein the compound that induces depletion of tumor-induced PMN-MDSCs is gemcitabine, fluorouracil, bindarit, PDE5 inhibitors, tadalafil, nitroaspirin, COX-2 inhibitors, ipilimumab, bevacizumab, celecoxib, a combination of sildenafil and tadalafil, N-hydroxy-L-arginine, N-acetyl cysteine (NAC), CpG oligodeoxy-nucleotides (ODN), Bardoxolone methyl (CDDO-Me), Withaferin A, monoclonal anti-Gr1 antibody, IL4Rα aptamer, peptibodies that target MDSC-membrane proteins, or an antibody against lymphocyte antigen 6 complex locus G6D (Ly6G).

29. The composition of claim 20, wherein the compound that induces depletion of tumor-induced PMN-MDSCs comprises an antibody against Ly6G.

30. The composition of claim 29, wherein the antibody against Ly6G is 1A8.

31. A recombinant modified vaccinia TianTan virus (rMVTT), the rMVTT comprising a deletion of the viral M1L-K2L genes from a vaccinia TianTan virus (VTT) and further comprising two or more heterologous polynucleotides that replace the deleted viral M1L-K2L genes.

32. The rMVTT according to claim 31, wherein one of the two or more heterologous polynucleotides comprises a heterologous polynucleotide encoding a fluorescent protein.

33. The rMVTT according to claim 32, wherein the fluorescent protein is HcRed or green fluorescent protein.

34. The rMVTT according claim 31, wherein one of the two or more heterologous polynucleotides comprises a heterologous polynucleotide encoding a capsid protein of a heterologous virus.

35. The rMVTT according to claim 34, wherein the capsid protein of the heterologous virus is p24 antigen (p24) of human immunodeficiency virus (HIV).

36. The rMVTT of claim 31, wherein one of the two or more heterologous polynucleotides are under the control of a synapsin promoter (pSYN) and another one of the two or more heterologous polynucleotides are under the control of an H5 promoter (pH5).

37. The rMVTT of claim 31, wherein one of the two or more heterologous polynucleotides comprises a heterologous polynucleotide encoding p24 of HIV under the control of pSYN and another one of the two or more heterologous polynucleotides comprises a heterologous polynucleotide encoding HcRed under the control of pH5.
